(19) United States
(12) Patent Application Publication
(10) Pub. No.: US 2025/0384963 A1
(43) Pub. Date: Dec. 18, 2025

(54) MOLECULAR METADATA

(71) Applicant: Othram, Inc., The Woodlands, TX (US)

(72) Inventors: David A. Mittelman, The Woodlands, TX (US); Lee Baker, The Woodlands, TX (US); John W. Fondon, III, The Woodlands, TX (US); Steven Bates, The Woodlands, TX (US); Steve W. Lockless, College Station, TX (US)

(73) Assignee: Othram, Inc., The Woodlands, TX (US)

(21) Appl. No.: 19/234,106

(22) Filed: Jun. 10, 2025

Related U.S. Application Data
(60) Provisional application No. 63/659,692, filed on Jun. 13, 2024.

(51) Int. Cl. G16B 40/10

(52) U.S. Cl. CPC G16B 40/10

(57) ABSTRACT

In some embodiments, a method for verifying genetic samples using contextual metadata can include receiving at least one genetic sample, identifying one or more contextual parameters associated with the at least one genetic sample, obtaining one or more etches selected from a set of etches to encode the one or more contextual parameters, and pooling the one or more etches with the at least one genetic sample in a sample environment, wherein pooling prevents binding of the one or more etches to the at least one genetic sample.

### CROSS-REFERENCE TO RELATED APPLICATIONS

This application claims the benefit of and priority to U.S. Provisional Application No. 63/659,692, filed Jun. 13, 2024, which is incorporated by reference herein in its entirety for all purposes.

### BACKGROUND

The present disclosure relates to the identification, verification, and protection of genetic data.

### SUMMARY OF DISCLOSURE

One embodiment relates to a method for verifying genetic samples using contextual metadata. In some embodiments, the method can include receiving at least one genetic sample. In some embodiments, the method can include identifying one or more contextual parameters associated with the at least one genetic sample. In some embodiments, the method can include obtaining one or more etches selected from a set of etches to encode the one or more contextual parameters. In some embodiments, the method can include pooling the one or more etches with the at least one genetic sample in a sample environment, wherein pooling prevents binding of the one or more etches to the at least one genetic sample.

In some embodiments, the at least one genetic sample includes a target genetic sample of an organism, and the method further includes receiving, using the sample environment, the one or more etches pooled with the target genetic sample for sequencing, and decoding, based on a sequencing output, the one or more etches to identify metadata of the target genetic sample represented by the one or more contextual parameters. The method further includes executing an error-detection code on the sequencing output to detect at least one error associated with the metadata. The method further includes executing an error-correction code on the sequencing output to correct the at least one error.

In some embodiments, the set of etches is stored in a library, and the method further includes selecting, from the library, the one or more etches to encode the one or more contextual parameters based on an encoding scheme, wherein the encoding scheme assigns a combination of at least two etches to represent a corresponding contextual parameter, and combining the at least two etches to encode the corresponding contextual parameter as a non-interfering genetic sequence.

In some embodiments, the method further includes detecting a contamination event based on identifying an etch pooled in the sample environment that encodes a contextual parameter associated with a genetic sample different from the at least one genetic sample.

In some embodiments, the method further includes storing the one or more contextual parameters encoded by the one or more etches in association with a physical or digital record corresponding to the at least one genetic sample.

In some embodiments, the one or more contextual parameters include at least one capture event parameter indicating information related to capture or collection of the at least one genetic sample and at least one processing event parameter indicating information related to processing, handling, or preparation of the at least one genetic sample.

In some embodiments, the set of etches is stored in a library, the library maps a plurality of etches to corresponding metadata, and the method further includes determining the one or more contextual parameters by comparing decoded values of the one or more etches to the corresponding metadata.

In some embodiments, the method further includes the one or more contextual parameters encoded by the one or more etches to verify an identity of the at least one genetic sample based on comparing the one or more contextual parameters and expected metadata.

In some embodiments, the method further includes executing an error-detection code or an error-correction code on a sequencing output corresponding to the one or more etches to detect at least one error associated with interpretation of the one or more contextual parameters.

In some embodiments, the method includes flagging, responsive to multiplexed sequencing of a plurality of genetic samples comprising the at least one genetic sample, an unexpected metadata combination associated with one or more of the plurality of genetic samples, and resolving the unexpected metadata combination using one or more resolution techniques.

In some embodiments, the method further includes validating the one or more contextual parameters by applying a threshold to sequencing read counts associated with each of the one or more etches, and a contextual parameter is identified based on detecting a combination of sequences with read counts satisfying the threshold.

One embodiment relates to a system for verifying genetic samples using contextual metadata. In some implementations, the system can include a set of etches and an encoding system. In some embodiments, the encoding system can be configured to receive at least one genetic sample. In some embodiments, the encoding system can be configured to identify one or more contextual parameters associated with the at least one genetic sample. In some embodiments, the encoding system can be configured to obtain one or more etches selected from set of etches to encode the one or more contextual parameters. In some embodiments, the encoding system can be configured to pool the one or more etches with the at least one genetic sample in a sample environment, wherein pooling prevents binding of the one or more etches to the at least one genetic sample.

In some embodiments, the at least one genetic sample includes a target genetic sample of an organism, and the system further includes a readout system configured to receive, using the sample environment, the one or more etches pooled with the target genetic sample for sequencing. The system can include a decoding system configured to decode, based on a sequencing output, the one or more etches to identify metadata of the target genetic sample represented by the one or more contextual parameter, decode, based on a sequencing output, the one or more etches to identify metadata of the target genetic sample represented by the one or more contextual parameters, execute an error-detection code on the sequencing output to detect at least one error associated with the metadata, and execute an error-correction code on the sequencing output to correct the at least one error.

In some embodiments, the set of etches is stored in a library, and the encoding system is further configured to select, from the library, the one or more etches to encode the one or more contextual parameters based on an encoding scheme, wherein the encoding scheme assigns a combination of at least two etches to represent a corresponding contextual parameter; and combine the at least two etches to encode the corresponding contextual parameter as a non-interfering genetic sequence.

In some embodiments, the system further includes a decoding system configured to detect a contamination event based on identifying an etch pooled in the sample environment that encodes a contextual parameter associated with a genetic sample different from the at least one genetic sample.

In some embodiments, the encoding system is further configured to store the one or more contextual parameters encoded by the one or more etches in association with a physical or digital record corresponding to the at least one genetic sample.

In some embodiments, the one or more contextual parameters include at least one capture event parameter indicating information related to capture or collection of the at least one genetic sample and at least one processing event parameter indicating information related to processing, handling, or preparation of the at least one genetic sample.

In some embodiments, the set of etches is stored in a library, the library maps a plurality of etches to corresponding metadata, and the system further includes a decoding system configured to determine the one or more contextual parameters by comparing decoded values of the one or more etches to the corresponding metadata.

In some embodiments, the system further includes a decoding system configured to use the one or more contextual parameters encoded by the one or more etches to verify an identity of the at least one genetic sample based on comparing the one or more contextual parameters and expected metadata.

One embodiment relates to a kit for verifying genetic samples using contextual metadata. The kit can include a predefined set of etches configured to encode one or more contextual parameters associated with at least one genetic sample. The kit can include a decoding reference database including mappings between the predefined set of etches and corresponding metadata values of the one or more contextual parameters.

One embodiment relates to a composition. The composition can include at least one genetic sample. The composition can include one or more etches selected to encode one or more contextual parameters associated with at least one genetic sample, wherein the one or more etches are non-covalently pooled with the at least one genetic sample in a sample environment.

One embodiment relates to a method for generating encoded genetic metadata, and including the encoded genetic metadata with a target genetic sample of an organism in a sample environment. In some embodiments, the method includes receiving the target genetic sample of the organism corresponding with capture event parameters. The method can further include identifying one or more processing event parameters for processing the target genetic sample. In some embodiments, the method can further include generating or obtaining or identifying encoded genetic metadata of the target genetic sample. The encoded genetic metadata can include one or more etches. In some embodiments, generating or obtaining or identifying the encoded genetic metadata can include encoding data of at least one of the capture event parameters or at least one of the processing event parameters as the one or more etches of the encoded genetic metadata and pooling the one or more etches of the encoded genetic metadata with the target genetic sample. In some embodiments, pooling includes including the one or more etches of the encoded genetic metadata with the target genetic sample in the sample environment. The method can further include providing the target genetic sample and encoded genetic metadata. In some embodiments, pooling maintains the target genetic sample in a processable or genetic-readable format.

In some embodiments, the target genetic sample of the organism includes DNA of a human organism, and wherein the one or more etches of the encoded genetic metadata include at least one non-human or non-interfering sequence.

In some embodiments, the one or more etches of the encoded genetic metadata diverge from a reference genome of the organism.

In some embodiments, the one or more capture event parameters includes one or more from a group consisting of a crime scene location, an officer identity, a suspect identity, a victim identity, a time, a timestamp of capture, a crime scene parameter, a unique reference number, a case number, a chain of custody, a crime type, and witness statements.

In some embodiments, the one or processing event parameters includes one or more from a group consisting of processing instructions, sequencing options, testing protocols, preservatives, unique reference numbers, case numbers, operator identity, training records, processing locations, times, timestamps of processing, quality assurance checks, compliance standards, calibration data, equipment use data, consumables and reagents tracking data, processing results, error logs, and data integrity measures.

In some embodiments, the sample environment including the target genetic sample and the encoded genetic metadata is a physical environment and a virtual environment.

In some embodiments, the physical environment is a sample container, and wherein the virtual environment is an electronic storage device.

In some embodiments, the organism is a human, and in some embodiments, the organism is a non-human organism.

In some embodiments, providing further includes providing the encoded genetic metadata and target genetic sample to an evidentiary system or a healthcare system.

In some embodiments, the method further includes receiving, by one or more processing circuits, data corresponding to the generation of the encoded genetic metadata, and storing, by the one or more processing circuits, the data corresponding the generation of the encoded genetic metadata in an electronic storage environment.

In some embodiments, the method further includes determining, by the one or more processing circuits and based on the stored data corresponding to the generation of the encoded genetic metadata, an update to the genetic metadata based on comparing data including the encoded genetic metadata to new data, generating updated encoded genetic metadata, wherein generating includes combining two or more etches stored in a predefined library to represent the new data, and updating, by the one or more processing circuits, the stored data to represent the new data.

In some embodiments, generating the encoded genetic metadata further includes synthesizing or obtaining or identifying two or more etches diverging from a human reference genome, and combining at least two of the two or more etches to represent at least one of the capture event parameters or processing event parameters as at least one non-human or non-interfering genetic sequence.

In some embodiments, generating the encoded genetic metadata further includes implementing an encoding scheme, and wherein the encoding scheme includes one or more from the group consisting of Manchester encoding, Differential encoding, Non-Return-to-Zero Inverted (NRZI) encoding, Pulse-code modulation (PCM), Binary Phase Shift Keying (BPSK), Miller encoding, 8b/10b encoding, 6b/8b encoding, binary-to-text encoding, one-hot encoding, label encoding, character encoding, HTML encoding, URL encoding, Unicode encoding, Base64 encoding, Hex encoding, ASCII encoding, and hashing encoding.

One embodiment relates to a system for generating encoded genetic metadata and including the encoded genetic metadata with a target genetic sample of an organism in a sample environment. The system can include a receiving system configured to receive the target genetic sample of the organism corresponding with capture event parameters and an identification system to identify one or more processing event parameters for processing the target genetic sample. In some embodiments, the system can include a generation system configured to generate or identify or obtain encoded genetic metadata of the target genetic sample including one or more etches by encoding data of at least one of the capture event parameters or at least one of the processing event parameters as the one or more etches of the encoded genetic metadata. The system can further include a pooling system to pool the encoded genetic metadata with the one or more genetic sequences of the target genetic sample by including the one or more etches of the encoded genetic metadata with the target genetic sample in the sample environment. In some embodiments, the system can include a providing system for providing the target genetic sample and encoded genetic metadata in a processable or genetic-readable format. In some embodiments, pooling can maintain the target genetic sample in a processable or genetic-readable format.

In some embodiments, the system can further include one or more processing circuits, wherein one or more of the receiving system, the identification system, the generation system or obtaining system or identification system, and the providing system utilize instructions executable by the one or more processing circuits.

In some embodiments, the one or more processing circuits are further configured to analyze the target genetic sample and encoded genetic metadata using the one or more processing event parameters, wherein the one or more processing circuits are configured to receive the target genetic sample and encoded genetic metadata via the sample environment, and sequence the target genetic sample and encoded genetic metadata.

In some embodiments, the one or more processing circuits are further configured to receive sequenced encoded genetic metadata from sequencing the target genetic sample and encoded genetic metadata, and in response to receiving the sequenced encoded genetic metadata, determine at least one error corresponding to the encoded genetic metadata by executing an error-detection code or an error-correction code, wherein the error-correction code analyzes the sequenced encoded genetic metadata to identify and output the at least one error.

In some embodiments, the error-detection code or the error-correction code includes one or more from a group consisting of: Hamming codes, Reed-Solomon codes, Low-Density Parity-Check (LDPC) codes, turbo codes, Cyclic Redundancy Check (CRC) codes, Bose-Chaudhuri-Hocquenghem (BCH) codes, RS232, Ethernet, TCP, UDP, Golay codes, Goppa codes, Viterbi decoders, multidimensional parity codes, checksum codes, hash codes, message authentication codes, alternant codes, AN codes, Berger codes, forward error correction codes, generalized minimum-distance codes, rank error-correction codes, and remote error indication codes.

In some embodiments, the one or more processing circuits are further configured to sequence the target genetic sample and encoded genetic metadata using a synthetic reference genome, wherein the synthetic reference genome includes a digital genetic sequence, and wherein the digital genetic sequence aligns or maps with the one or more etches of the encoded genetic metadata.

In some embodiments, the techniques described herein relate to a system, wherein the one or more processing circuits are further configured to compare the one or more etches of the sequenced encoded genetic metadata to a genetic reference dataset to determine at least one of the capture event parameters or processing event parameters encoded in the genetic material metadata, and determine the determined at least one capture event parameter or processing event parameter aligns with at least one expected capture event parameter or expected processing event parameter.

In some embodiments, the generation system is further configured to generate one or more reagents for amplifying the encoded genetic metadata, wherein generating includes synthesizing or obtaining or identifying one or more genetic primers or probes to selectively bind to at least one of the one or more etches of the encoded genetic metadata, and applying the one or more reagents to the encoded genetic metadata via the sample environment.

In some embodiments, the target genetic sample of the organism includes DNA of a human organism, and wherein the one or more etches of the encoded genetic metadata include at least one non-human or non-interfering sequence.

In some embodiments, the one or more etches of the encoded genetic metadata diverge from a reference genome of the organism.

In some embodiments, the one or more capture event parameters includes one or more from a group consisting of a crime scene location, an officer identity, a suspect identity, a victim identity, a time, a timestamp of capture, a crime scene parameter, a unique reference number, a case number, a chain of custody, a crime type, and witness statements.

In some embodiments, the one or processing event parameters includes one or more from a group consisting of processing instructions, sequencing options, testing protocols, preservatives, unique reference numbers, case numbers, operator identity, training records, processing locations, times, timestamps of processing, quality assurance checks, compliance standards, calibration data, equipment use data, consumables and reagents tracking data, processing results, error logs, and data integrity measures.

In some embodiments, the sample environment including the target genetic sample and the encoded genetic metadata is a physical environment and a virtual environment.

In some embodiments, the physical environment is a sample container, and in some embodiments, the virtual environment is an electronic storage device.

In some embodiments, the organism is a human, and in some embodiments, the organism is a non-human organism.

In some embodiments, the providing system is further configured to provide the encoded genetic metadata and target genetic sample to an evidentiary system or a healthcare system.

In some embodiments the receiving system is further configured to receive data corresponding to the generation of the encoded genetic metadata, and store the data corresponding to the generation of the encoded genetic metadata in an electronic storage environment.

One embodiment relates to one or more non-transitory computer-readable storage media (CRM) having instructions stored thereon that, when executed by a one or more processing circuits, cause the one or more processing circuits to receive a target genetic sample of an organism corresponding with capture event parameters. The one or more processing circuits can further identify one or more processing event parameters for processing the target genetic sample and generate encoded genetic metadata of the target genetic sample. In some embodiments, the encoded genetic metadata can include one or more etches. In some embodiments, the one or more processing circuits can generate or identify or obtain the encoded genetic metadata by encoding data of at least one of the capture event parameters or at least one of the processing event parameters as the one or more etches of the encoded genetic metadata. In some embodiments, the one or more processing circuits can provide the target genetic sample and encoded genetic metadata. In some embodiments, the one or more etches of the encoded genetic metadata can be pooled with the target genetic sample in a sample environment. In some embodiments, the sample environment can maintain the target genetic sample in a processable or genetic-readable format.

In some embodiments, the one or more non-transitory CRM have additional instructions stored thereon that, when executed by the one or more processing circuits, cause the one or more processing circuits to sequence pooled contents of the sample environment including the one or more etches of the encoded genetic metadata and the target genetic sample.

One embodiment relate to a method of providing a sample environment for storing a target genetic sample of an organism with encoded genetic metadata associated with the target genetic sample. The method can include adding the target genetic sample of the organism to a sample environment. In some embodiments, the sample environment includes a container for storing genetic material. The method can further include adding one or more etches of the encoded genetic metadata to the sample environment. In some embodiments, adding includes including the one or more etches of the encoded genetic metadata in the sample environment unbonded with the target genetic sample and providing the sample environment including the one or more etches of the encoded genetic metadata and target genetic sample to a sequencing system.

In some embodiments, adding the one or more etches of the encoded genetic metadata to the sample environment further includes adding the one or more etches of the encoded genetic metadata to a metadata sample environment, and adding the one or more etches of the encoded genetic metadata from the metadata sample environment to the sample environment, wherein adding includes adding the one or more etches of the encoded genetic metadata separately from the one or more genetic sequences of the target genetic sample.

### BRIEF DESCRIPTION OF THE DRAWINGS

FIG. 1 is a block diagram depicting an example of a system for verifying genetic samples using contextual metadata, according to some implementations.

FIG. 2 is a flowchart for a method for verifying genetic samples using contextual metadata, according to some implementations.

FIG. 3 is a block diagram depicting an example of a system for data identification and data protection, according to some implementations.

FIG. 4 is a flowchart for a method for generating and incorporating encoded genetic metadata with a target genetic sample of an organism, according to some implementations.

FIGS. 5A-5D are block diagrams depicting an example of a sample environment for data identification and data protection, according to some implementations.

FIGS. 6A-6B are flowcharts for a method for generating and incorporating encoded genetic metadata with a target genetic sample of an organism, according to some implementations.

FIGS. 7A-7E are block diagrams depicting examples of genetic metadata and encoding, according to some implementations.

FIGS. 8A-8B are example illustrations depicting a system for receiving and storing genetic metadata, according to some implementations.

FIG. 9 is a block diagram illustrating an example computing system suitable for use in the various implementations described herein.

It will be recognized that some or all of the figures are schematic representations for purposes of illustration. The figures are provided for the purpose of illustrating one or more implementations, embodiments, or arrangements with the explicit understanding that they will not be used to limit the scope or the meaning of the claims.

### DETAILED DESCRIPTION

The present disclosure is directed to systems and methods for using molecular etches to identify, verify, and/or protect genetic data. The systems and methods described herein provide improvements in the security of genetic data by including contextual information or metadata associated with capture or processing of the genetic data with the genetic data itself (e.g., using etches pooled in a sample environment containing the genetic data). The inclusion of genetic metadata provides computational improvements in processing and/or sequencing of a target genetic sample by providing information to a processing or sequencing system to quickly, efficiently, and accurately identify data corresponding to the target genetic sample (e.g., case number, identifier, etc.) from an additional source other than the target genetic sample. As such, computing systems can use genetic metadata encoded using etches as a supplemental data source to quickly and accurately access case-related information associated with a genetic sample without relying on static tags or identifiers. For example, barcodes or unique identifiers can be appended to a sample for basic tracking or demultiplexing, but do not encode interpretable metadata associated with genetic sample that provides granular or contextual information beyond a sample identifier. In contrast, the systems and methods described herein can use molecular etches as an internal molecular information management system (IMIMS) that encodes contextual metadata associated with a genetic sample. Moreover, the metadata encoded by the etches can be decoded, validated, and compared against reference records to assess sample provenance or detect workflow errors or anomalies. Further, by pooling the etches with the genetic sample, computing systems can implement an improved and robust data access and error-checking protocol (e.g., using metadata encoded by the etches for validation, error identification, etc.), thereby providing data integrity and improving computational efficiency.

In particular, the systems and methods described herein generate and incorporate encoded genetic metadata with a target genetic sample without binding to, altering, or perturbing the target genetic sample (e.g., other than common alterations made during pre-processing, processing, PCR, sequencing, etc.—common alterations can include amplification, fragmentation, and purification steps). For example, in contrast to inline barcoding, the molecular etches described herein can be added to the genetic sample without binding to the DNA of the genetic sample itself. This protects a target genetic sample by avoiding data degradation that can occur when additional genetic material binds to a DNA sample, and preventing such binding or alterations (e.g., other than routine or expected alterations occurring during sequencing, such as alterations resulting from DNA marking, polymerase errors, or natural genetic variation, etc.) preserves the ability to effectively present genetic data related to the target genetic sample to a public audience (e.g., jury, grand jury) or as a public record (e.g., criminal court records, DNA databases, etc.). That is, by maintaining the integrity of the DNA sample while incorporating encoded genetic metadata, researchers, public audiences, and other entities can confidently analyze and interpret the genetic information obtained, providing an accurate and reliable indicator associated with a sample for downstream analyses. Additionally, this improves the reproducibility of genetic analyses, as the integrity of the sample is maintained throughout a lifecycle of the sample. Moreover, the capability to store genetic metadata alongside the sample without modifying or binding to the underlying target genetic sequences safeguards the original genetic information from unintentional alterations, further bolstering the reliability and traceability of genetic data in forensic and clinical settings. The separation between metadata and associated DNA sample material further provides technical improvements by improving data integrity and preventing interference between raw genetic data and associated metadata.

Moreover, the systems and methods herein include the use of non-human synthetic etches that do not interfere with the processing or sequencing of the target genetic sample. The use of non-human etches (or other sequences that do not align with a reference genome, such as a standard reference genome of the organism providing the DNA sample) in some implementations can facilitate that the metadata does not contaminate or alter the genetic information associated with the target sample, thereby promoting data integrity and reducing processing errors by maintaining the purity and accuracy of genetic data before genetic analysis. Utilizing non-human etches as metadata also facilitates the differentiation of data types during subsequent analytical processes, reducing computational complexity and increasing computing efficiency, which can improve the clarity and efficiency of data handling in genetic sequencing environments. Further, the systems and methods presented herein reduce the risk of cross-contamination and minimize errors associated with sample processing by providing more streamlined integrations of genetic metadata into existing genomic databases and software, thereby improving data management technologies by facilitating more accurate and computationally efficient management and retrieval of genetic data. Specifically, the systems and methods disclosed herein collectively improve the robustness of the sequencing workflows and contribute to more accurate genetic interpretations of sample genetic data.

Furthermore, the systems and methods disclosed herein provide the advantage of selecting from a library of pre-defined non-human etches when encoding genetic metadata. This approach avoids synthesizing custom etches when processing each sample, significantly reducing the computing time and computational resource expenditure (e.g., processing loads, storage space, etc.) associated with the creation of synthetic etches. The implementations including a library with predefined etches provide for quick integration and implementation of genetic metadata, which improves the speed and efficiency of encoding process and facilitates more consistent and scalable metadata applications across different genetic samples. This provides technological advantages including promoting computational interoperability among different genetic databases and facilitating easier sharing of metadata across platforms, thereby improving computing speeds and efficiency. Additionally, the use of a pre-defined library simplifies the maintenance and updates of metadata standards, which can be quickly and automatically updated by the systems and methods described herein based on ongoing advancements in genetic technologies and related methodologies. Thus, the systems and methods disclosed herein increase genetic data processing speeds, in addition to strengthening the overall security and utility of genetic information management systems.

As used herein, the terms “a genetic sequence,” “genetic sequences,” “genetic material,” and the like can refer to an ordered series of nucleotides that include a segment of DNA or RNA, represented by specific chemical symbols: adenine (A), cytosine (C), guanine (G), and thymine (T) for DNA, and uracil (U) in place of thymine for RNA. These sequences can physically exist as part of a nucleic acid molecule (e.g., including a target genetic sample) or can be represented virtually as a series of letters (e.g., A, C, G, T, U) in computational systems (e.g., in a database, etc.) for the purpose of genetic analysis or manipulation. Each of these chemicals, known as bases, participates in the storage and transmission of genetic information by pairing with a complementary base (A pairs with T or U, and C pairs with G) to form the structural backbone of the genetic material.

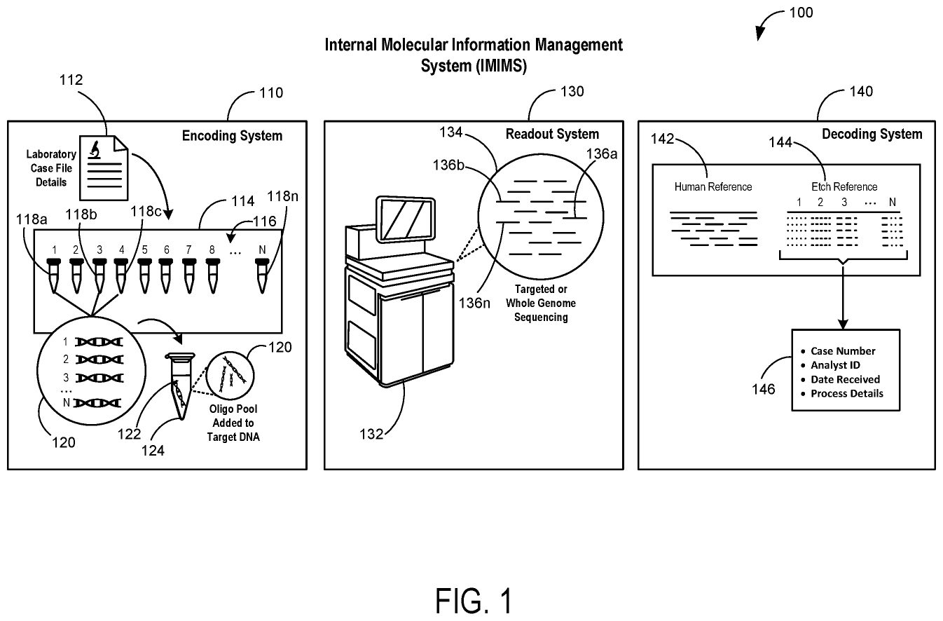

Referring to FIG. 1, a block diagram depicting an example of a system 100 for verifying genetic samples using contextual metadata is shown, according to some implementations. The system 100 can include an encoding system 110, a readout system 130, and a decoding system 140. The encoding system 110 can include a metadata input source 112, a library 114, and a sample container 124. The library 114 can include a set of etches 116, and the set of etches 116 can include etches 118a-118n (collectively, etches 118). One or more of the etches 118a-118n can be selected and included in selected etches 120 (e.g., an oligo pool). For example, the selected etches 120 can be pooled with a genetic sample 122 in the sample container 124. The readout system 130 can include a sequencing system 132 configured to sequence genetic material included in the sample container 124 to generate a sequencing output 134. The sequencing output 134 can include one or more read sequences 136a-136n (collectively, read sequences 136) corresponding to at least one of the selected etches 120 or the genetic sample 122. The decoding system 140 can include a human reference 142, an etch reference 144, and a metadata output 146.

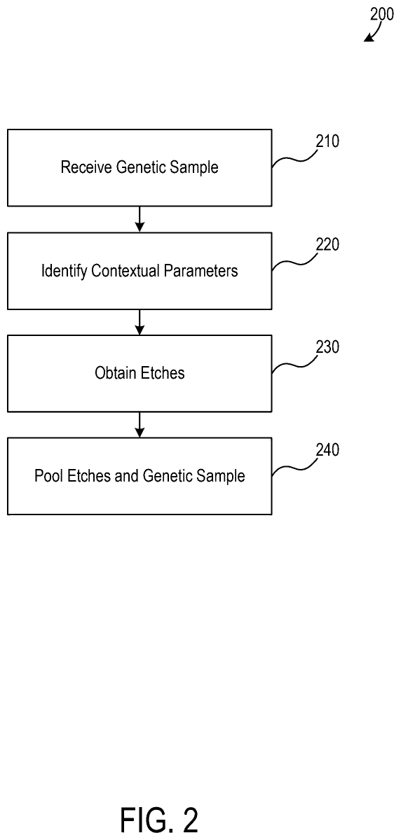

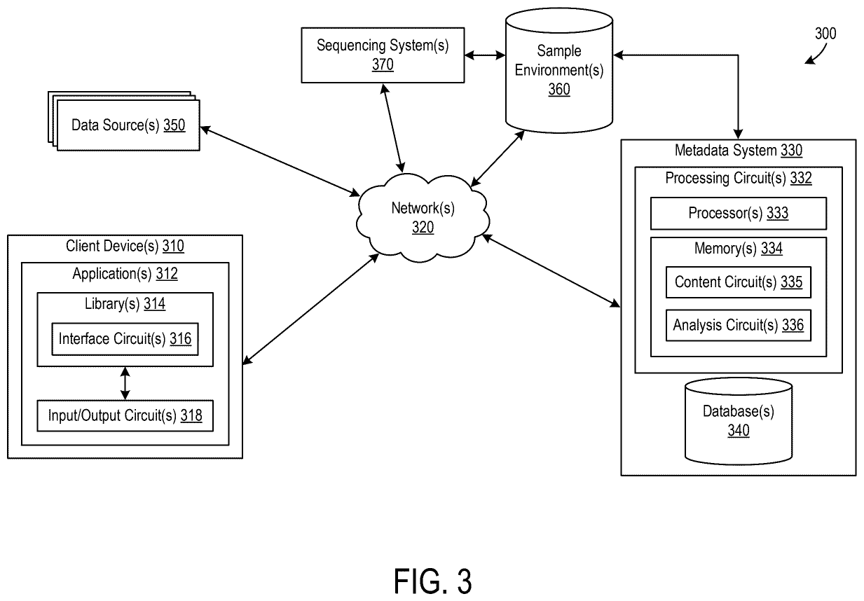

In some implementations, although the various components of FIG. 1 can be described in the singular form below (e.g., metadata input source 112, sample container 124, etc.), it should be understood that the system 100 can include two or more of any element described herein (e.g., two or more metadata input sources 112, sample containers 124, etc.). In some implementations, one or more of the systems, sub-systems, or components described with regard to FIG. 1 can correspond to (e.g., include the same or similar features and/or functionality as described regarding) one or more systems, sub-systems, or components described in FIGS. 2-9. For example, the metadata input source 112 of FIG. 1 can correspond to the input/output circuit 318 of FIG. 3, the library 114 can correspond to database 340 of FIG. 3, the encoding system 110 can correspond to the metadata system 330 or the analysis circuit 336 of FIG. 3, the sample container 124 can correspond to the sample environment 360 of FIG. 3, the readout system 130 or sequencing system 132 can correspond to the sequencing system 370 of FIG. 3, the decoding system 140 can correspond to the metadata system 330 or analysis circuit 336 of FIG. 3, and so on. One or more of the systems, sub-systems, or components of FIG. 1 (e.g., encoding system 110, readout system 130, decoding system 140, etc.) can include various combinations of hardware and/or software (e.g., one or more processors coupled with memory) configured or structured to execute or perform various operations described herein.

In some implementations, the encoding system 110 can include metadata input source 112. For example, the metadata input source 112 can be configured to receive or access contextual metadata associated with a genetic sample 122. The contextual metadata can include contextual information associated with capture, processing, handling, storage, transportation, or other lifecycle events of the genetic sample 122. For example, metadata input source 112 can receive or identify one or more capture event parameters (e.g., case number, collection timestamp, geographic location, etc.), one or more processing event parameters (e.g., sequencing method, reagent data, processing timestamp, equipment configuration, etc.), and/or one or more additional contextual parameters associated with genetic sample 122 (e.g., custody records, storage conditions, transport routes, environmental conditions, etc.). In some examples, the metadata input source 112 can include, or be communicatively coupled to, one or more data storages, data entry interfaces (e.g., graphical user interfaces), laboratory information management systems (LIMS), document scanners, or computing devices configured to provide case data, laboratory forms, or other metadata inputs associated with the genetic sample 122. In some examples, the metadata input source 112 can be configured to structure or standardize the received metadata for downstream encoding. For example, the metadata input source 112 can convert input metadata into a predefined data format or schema used to select corresponding etches 118 from the library 114.

In some implementations, the encoding system 110 can include a library 114. For example, the library 114 can be configured to store the set of etches 116 including one or more individual etches 118a-118n (e.g., etches). The etches 118 can include predefined, non-interfering oligonucleotides selected to represent contextual metadata associated with a genetic sample 122. For example, one or more of the etches 118 stored in the library can be selected or configured to prevent interference with human reference 142. In some examples, the etches 118 can be generated or selected such that each etch or group of etch includes a sufficient number of unique base positions and satisfies a predefined minimum edit distance relative to other etches in the set. That is, the library 114 can be constructed to reduce the likelihood that sequencing or amplification errors would result in misclassification of etches during downstream processing. In some implementations, the sequencing system 132 can achieve read depths sufficient to detect etches present at low concentrations (e.g., less than 1% of total input DNA), thereby supporting detection of sample contamination or mislabeling based on presence or absence of one or more selected etches 120 in a sequencing output 134.

In some examples, the encoding system 110 can select one or more etches 118 from the library 114 to encode a corresponding metadata value (e.g., contextual parameter) associated with capture or processing of the genetic sample 122. For example, the encoding system 110 can select etch 118a to encode a representation of signature of an analyst associated with processing of the genetic sample 122. In some examples, the encoding system 110 can combine two or more etches 118 stored in the library 114 to encode a metadata value or contextual parameter according to an encoding scheme. For example, the encoding system 110 can select etch 118b, etch 118c, and/or additional etches to encode a representation of a date (e.g., day, month, year, etc.) during which the genetic sample 122 was captured or processed. The encoding system 110 can provide the genetic sample 122 along with one or more selected etches 120 for further processing (e.g., sequencing).

In some implementations, the encoding system 110 can include a sample container 124 configured to pool the genetic sample 122 and selected etches 120. For example, the sample container 124 can include or refer to a sample environment, such as a physical reaction vessel (e.g., a tube, cartridge, or well plate) suitable for library preparation or next-gen sequencing (NGS) workflows. In some examples, information associated with the genetic material pooled in the sample container 124 can be stored in a digital environment (e.g., a database, a laboratory information management system, etc.) configured to track metadata associated with the pooled genetic material. For example, the term “sample environment,” as used herein, can include or refer to a physical and/or virtual context in which a target genetic sample is associated with etches that encode metadata. The sample environment may include a physical container such as a reaction vessel, test tube, cartridge, or sequencing well in which both the sample DNA and the synthetic etches are pooled for processing, without chemical or covalent bonding between them (e.g., sample container 124). Alternatively or additionally, the sample environment may include a virtual or digital representation of the sample and associated metadata, such as a record in a database or laboratory information management system. In some implementations, the sample environment includes both physical and digital components to facilitate tracking, analysis, and interpretation of a genetic sample and associated metadata throughout a lifecycle of the genetic sample. In some implementations, the sample environment maintains the genetic material and molecular etches in a format that is compatible with downstream analysis, such as sequencing, amplification, or quality control, and supports secure and reproducible data handling. In some examples, the selected etches 120 can be introduced to the sample container 124 including the genetic sample 122 in a non-covalent manner (e.g., to prevent covalent binding between the selected etches 120 and the genetic sample 122). For example, the sample container 124 can pool the selected etches 120 and genetic sample 122 such that the selected etches 120 are not physically bound to genetic sample 122 but remain present, detectable, and analytically distinguishable during of sequencing of the combined pooled genetic material included in the sample container 124.

In some implementations, the readout system 130 can include a sequencing system 132. For example, the sequencing system 132 can be configured to sequence the pooled genetic contents of the sample container 124 and identify read sequences 136 corresponding to the genetic sample 122 and/or the selected etches 120. The sequencing system 132 can include one or more sequencing instruments, platforms, or pipelines configured to support high-throughput sequencing technologies (e.g., massively parallel sequencing (MPS), next-generation sequencing (NGS), nanopore sequencing, or other suitable sequencing methodologies). In some examples, the sequencing system 132 can implement a read protocol and sequencing chemistry configured to resolve short DNA fragments without PCR-based incorporation, and in some examples, the sequencing system 132 can amplification-based tagging strategies to increase detection sensitivity for low-abundance sequences. For example, the selected etches 120 can be directly sequenced as non-amplified input during library preparation and remain detectable during readout, or can be processed through adapter ligation and PCR amplification to increase detectability. In some implementations, the sequencing system 132 can perform base calling to determine the nucleotide content of each read and generate a sequencing output 134 including one or more read sequences 136. The sequencing output 134 can include target read sequences (e.g., read sequence 136a) corresponding to the genetic sample 122 and non-target read sequences (e.g., read sequence 136b) corresponding to the selected etches 120. In some implementations, the sequencing output 134 can be generated in a format compatible with downstream alignment and analysis workflows (e.g., using as FASTQ or BAM format). The readout system 130 can provide the sequencing output 134 including the read sequences 136 to the decoding system 140 for further processing (e.g., decoding or post-sequencing interpretation).

In some implementations, the sequencing system 132 can be configured to co-sequence the selected etches 120 and the genetic sample 122 using a unified library preparation workflow. For example, the etches 120 can be introduced into the sample container 124 as non-covalently mixed inputs (e.g., physically separable blunt-ended or adapter-ligated oligonucleotides) that are not chemically modified or conjugated to the genomic material of the genetic sample 122. The sequencing system 132 can include reagents, adapters, or processing components compatible with both the selected etches 120 and the genetic sample 122 (e.g., components configured for pooling, ligation, amplification, and sequencing within a shared workflow). As a result, the etches 120 and the genetic sample 122 can pass through library preparation and sequencing as a combined input, while remaining analytically distinguishable in the sequencing output 134 (e.g., via mapping read sequence 136b to an etch reference 144 and read sequence 136a to a human reference 142).

In some implementations, the decoding system 140 can include a human reference 142, an etch reference 144, and a metadata output 146. The human reference 142 can include one or more reference genomes or datasets used to align and interpret read sequences 136 corresponding to the genetic sample 122. The etch reference 144 can include a reference database (e.g., decoding reference database) that stores information associated with etches 118 used for encoding and/or decoding contextual metadata (e.g., mappings of etches stored in the library 114 and corresponding metadata values represented by the stored etches). In some examples, the decoding system 140 can identify one or more etches present in the sequencing output 134 by comparing read sequences 136 to the etch reference 144, and can determine whether one or more identified etches in the sample container 124 correspond to an expected metadata value. For example, the decoding system 140 can evaluate the presence, absence, or composition of the selected etches 120 to validate provenance or integrity of the genetic sample 122. For example, the decoding system 140 can execute an error-detection code to detect a mismatch between expected and observed etch combinations (e.g., identifying extraneous or missing etches), which can indicate a sample swap, labeling error, processing error, or contamination event. In some implementations, the decoding system 140 can generate a metadata output 146 including one or more decoded contextual metadata values based on the identification of etches 120. In some examples, the decoding system 140 can execute an error-correction code to produce a metadata output 146 that corrects an error detected during sequencing. The metadata output 146 can be provided in association with the genetic sample 122 to facilitate downstream tracking, review, or reporting, as further described herein.

Referring now to FIG. 2, a flowchart for a method 200 for verifying genetic samples using contextual metadata is shown, according to some implementations, according to some implementations. At least the system 100 of FIG. 1 and/or additional components, systems, or sub-systems described herein (e.g., metadata system 330 of FIG. 3) can be configured to perform method 200. In broad overview of method 200, block 210 can include receiving a target genetic sample. Block 220 can include identifying contextual parameters. Block 230 can include obtaining etches. Block 240 can include pooling the etches and genetic sample. In some implementations, one or more of the steps or blocks of method 200 can be executed sequentially or in parallel, and one or more steps or blocks can be removed, added, reordered, and/or otherwise modified.

In some implementations, block 210 can include receiving a genetic sample. For example, at block 210, an encoding system (e.g., encoding system 110) can receive, obtain, and/or otherwise identify at least one genetic sample (e.g., genetic sample 122). Receiving the at least one genetic sample can include the encoding system 110 identifying or acquiring a biological specimen of an organism (e.g., hair, buccal swab, tissue, blood, etc.). In some implementations, receiving the genetic sample can include extracting genetic material from the biological specimen and storing the extracted genetic material for subsequent processing. For example, the encoding system 110 can extract genetic material (e.g., nucleic acids) from the biological specimen (e.g., using a silica column-based or magnetic bead-based extraction kit), identify the quantity of the extracted genetic material (e.g., using a fluorometric assay kit), and store the extracted genetic material temporarily or transfer the genetic material into a sample environment (e.g., sample container 124) for subsequent processing. In some implementations, receiving the at least one genetic sample can include the encoding system 110 initiating a sample intake process that associates the received genetic sample with metadata or contextual parameters indicating capture events or processing events associated with the genetic sample (e.g., a case number, collection date, specimen type, handling conditions, etc.).

In some implementations, block 220 can include identifying contextual parameters. For example, at block 220, the encoding system (e.g., encoding system 110) can identify, determine, and/or otherwise identify one or more contextual parameters associated with the at least one genetic sample. Identifying the contextual parameters can include the encoding system 110 accessing, receiving, and/or otherwise retrieving metadata from one or more data sources (e.g., laboratory information management systems, graphical user interfaces, or scanned documentation) associated with the intake or processing of the genetic sample 122 using metadata input source 112. For example, the metadata input source 112 can identify contextual parameters including a case number, a sample collection timestamp, a geographic collection location, a timestamp, an instrumentation identifier, or other metadata associated with capture, handling, or processing of the genetic sample 122. The contextual parameters can include and/or be associated with metadata values configured for downstream interpretation or quality assurance. For example, the metadata input source 112 can identify one or more contextual parameters indicating a stage of forensic processing (e.g., whether the genetic sample 122 is undergoing initial analysis or reanalysis), the personnel or laboratory responsible for processing, the analytical workflow applied or scheduled (e.g., extraction-only, STR profiling, or genome-wide sequencing), the presence of internal quality controls (e.g., whether the sample was co-processed with a negative or positive control), or other metadata indicating historical values, current values, or expected values associated with capture, handing, or processing of the genetic sample 122. In some examples, the contextual parameters identified at block 220 can be used to select one or more etches 118 from the library 114 to encode contextual information (e.g., metadata) of the genetic sample 122, as further described with regard to block 230.

In some implementations, block 230 can include obtaining etches. For example, at block 230, the encoding system (e.g., encoding system 110) can obtain, retrieve, generate, and/or otherwise acquire one or more etches (e.g., etches 118) selected from set of etches (e.g., set of etches 116) to encode the one or more contextual parameters. In some examples, an etch can correspond to a discrete metadata value or contextual parameter (e.g., etch 118a corresponding to an analyst identifier, etc.), and in some examples, a combination of multiple etches can be selected to represent a metadata value or contextual parameter (e.g., etch 118b, etch 118c, and/or additional etches to encode a representation of a date). The etches 118 obtained by the encoding system 110 can include short, non-interfering double-stranded DNA fragments (e.g., 150 bp) that satisfy predefined quality constraints (e.g., minimum edit distance, absence from genomic and microbial databases, uniform base composition, etc.), and that have been validated to support high-fidelity readout by satisfying one or more constraints during library preparation or sequencing (e.g., etch uniqueness, synthesis purity, error tolerance, etc.). In some examples, one or more of the etches 118 obtained by the encoding system 110 can be grouped (e.g., into an oligo pool including selected etches 120) and prepared for subsequent pooling with the genetic sample 122, as further described with regard to block 240.

In some implementations, block 240 can include pooling the etches and genetic sample. For example, at block 240, the encoding system (e.g., encoding system 110) can pool the one or more etches with the at least one genetic sample in a sample environment (e.g., sample container 124). In some implementations, pooling can prevent binding of the one or more etches to the at least one genetic sample. For example, pooling the selected etches 120 with the genetic sample 122 can include introducing the selected etches 120 in a non-covalent manner, such that the selected etches 120 remain physically separable and analytically distinguishable from the endogenous nucleic acids of the genetic sample 122. In some examples, the selected etches 120 can be added to the sample container 124 prior to library preparation steps such as fragmentation, end-repair, adapter ligation, or amplification. For example, the etches 120 can be added in picogram quantities (e.g., 30-150 pg total input) relative to nanogram quantities of genomic DNA to preserve the original composition of the genetic sample 122 while facilitating low-level detection of molecular etches. In some examples, resulting pooled sample included in the sample container 124 can be processed in accordance with library preparation workflows compatible with downstream sequencing systems (e.g., Illumina or Oxford Nanopore), in which both the genetic sample 122 and the etches 120 are processed jointly without chemical modification or conjugation. In some implementations, pooling can be performed manually or using automated systems, and the quantity and identity of the pooled etches can be recorded for subsequent decoding.

In some implementations, the set of etches can be stored in a library (e.g., library 114). In some implementations, the method 200 can include selecting, from the library, the one or more etches to encode the one or more contextual parameters based on an encoding scheme, and the encoding scheme can assign a combination of at least two etches to represent a corresponding contextual parameter. For example, the encoding system (e.g., encoding system 110) can select a first group of etches to encode a calendar day value, a second group of etches to encode a month value, and a third group of etches to encode a year value. For example, a component of an overall metadata value (e.g., a date value including a day, month, and year) can be represented by a distinct combination of two or more etches 118 selected from the library 114 based on a predefined encoding scheme. In some implementations, the method 200 can include combining the at least two etches to encode the corresponding contextual parameter as a non-interfering genetic sequence. For example, combining can include pooling the etches in a shared oligo pool (e.g., selected etches 120) prior to pooling the etches with the genetic sample 122 in the sample container 124. In some examples, combining can include pooling the etches such that the combination of the etches does not interfere with or map to human reference 142 and maintains sufficient sequence distance from other etches in the library 114.

In some implementations, the method 200 can include detecting a contamination event based on identifying an etch pooled in the sample environment that encodes a contextual parameter associated with a genetic sample different from the at least one genetic sample. For example, detecting can include identifying, via the decoding system (e.g., decoding system 140), one or more etches 118 in the sequencing output 134 that correspond to a contextual parameter not expected for the genetic sample 122. For example, in preparation for sequencing, the encoding system can store metadata values associated with genetic sample and encoded by the etches, and in response to sequencing, the decoding system 140 can compare read sequences 136 identified from sequencing the pooled contents to the etch reference 144 to detect extraneous etches that map to a different analyst signature, case number, sample identifier, processing timestamp, or other contextual parameter that was not stored for the genetic sample 122. For example, detecting the presence of an unexpected etch can indicate a potential contamination, sample swap, or labeling error during handling or processing. In some examples, in response to detection of the contamination event, the decoding system 140 can flag the contextual mismatch as an error or potential error and output a corresponding metadata output 146 that reflects the discrepancy for downstream review, remediation, or rejection of the affected sample.

In some implementations, the method 200 can include storing the one or more contextual parameters encoded by the one or more etches in association with a physical or digital record corresponding to the at least one genetic sample. For example, in preparation for sequencing, the encoding system (e.g., encoding system 110) can generate or update a metadata record (e.g., a physical record such as a worksheet or laboratory log, a digital record such as an entry in database, etc.) that links the genetic sample 122 to the contextual parameters selected for encoding (e.g., via metadata input source 112). For example, the contextual parameters encoded for the genetic sample can be stored in a digital environment such as a laboratory information management system (LIMS) or other case management system, and can include metadata fields indicating the sample identifier, selected contextual parameters, corresponding etches, or a mapping to the etch reference 144. The physical or digital record can be referenced by the decoding system 140 to verify consistency between expected metadata and results derived from the sequencing output 134 (e.g., metadata identified during sequencing).

In some implementations, the one or more contextual parameters include at least one capture event parameter indicating information related to capture or collection of the at least one genetic sample. For example, capture event parameters can include a case number, a field collection timestamp, a collection site or geographic location, a collection method (e.g., buccal swab, blood draw, tissue biopsy), a sample type (e.g., whole blood, epithelial cells), or an identifier of the individual or team responsible for sample collection. In some examples, a capture event parameter can be used to trace sample provenance or assess chain-of-custody in forensic or clinical applications. In some implementations, the one or more contextual parameters include at least one processing event parameter indicating information related to processing, handling, or preparation of the at least one genetic sample. For example, processing event parameters can include the name or identifier of the processing laboratory, the processing date, the operator or technician identifier, the type of analysis to be performed (e.g., short tandem repeat profiling, targeted sequencing, whole-genome sequencing), the reagent batch identifier, or the processing instrument used. In some examples, a processing event parameter can be used to associate the genetic sample with laboratory workflow milestones or support downstream quality control and audit processes. In some implementations, the set of etches can include a plurality of etches. For example, a plurality of molecular etches can include multiple non-coding DNA sequences configured to encode a discrete metadata value or a portion of a contextual parameter. In some examples, molecular etches can be configured to avoid homology to known genomic sequences and can satisfy constraints for orthogonality, edit distance, base composition, and compatibility with sequencing protocols. The molecular etches can be stored in a library (e.g., library 114) and selectively pooled to form a metadata-encoding oligo pool (e.g., selected etches 120) that is added to the genetic sample 122 for joint or combined processing.

In some implementations, the at least one genetic sample includes a target genetic sample of an organism. For example, the target genetic sample can include genomic DNA extracted from a biological specimen collected from a human or non-human subject (e.g., forensic reference sample, clinical isolate, environmental trace sample, or biological research specimen). In some examples, the target genetic sample can be processed (e.g., using sequencing system 132) to determine individual identity, genetic variation, or other biological characteristics associated with the target genetic sample. In some implementations, the method 200 can include receiving, using the sample environment, the one or more etches pooled with the target genetic sample for sequencing. For example, the sample container 124 can receive the metadata-encoding oligo pool (e.g., selected etches 120) along with the extracted genetic material of the target genetic sample, and the pooled contents can then be processed in accordance with a unified library preparation protocol compatible with downstream sequencing workflows. In some examples, library preparation can be performed using a PCR-free protocol (e.g., direct ligation of adapters) to preserve the integrity of the target genetic material and reduce amplification bias, and in some example, PCR amplification can be selectively applied to increase detection sensitivity for etches introduced at low abundance.

In some implementations, the method 200 can include decoding, based on a sequencing output, the one or more etches to identify metadata of the target genetic sample represented by the one or more contextual parameters. For example, decoding can include comparing read sequences corresponding to the etches against a reference set of molecular etches (e.g., etch reference 144) to identify metadata values encoded in the oligo pool. In some examples, decoding can include the decoding system 140 referencing an encoding scheme, mapping detected etches or read sequences to corresponding metadata entries stored in a physical or digital record based on the encoding scheme, and verifying whether the identified metadata matches or fails to match expected values stored for the genetic sample 122.

In some implementations, the method 200 can include executing an error-detection code on the sequencing output to detect at least one error associated with the metadata. For example, executing the error-detection code can include the decoding system 140 using a cyclic redundancy check (CRC), parity bit scheme, Hamming code, or other error-detection technique, tool, or algorithm to determine whether the identified metadata includes inconsistencies or invalid entries based on the applied encoding scheme. In some implementations, the method 200 can include executing an error-correction code on the sequencing output to correct the at least one error. For example, executing the error-correction code can include the decoding system 140 applying Reed-Solomon coding, BCH coding, a convolutional code, or other forward error correction technique, tool, or algorithm to recover one or more corrupted or missing values from the read sequence corresponding to the etches and produce a corrected sequencing output. In some examples, executing the error-correction code can include scoring multiple candidate reads or sequences for likelihood based on factors (e.g., base quality metrics, redundancy in the etch, etc.) and/or selecting a probable metadata value associated with the candidate reads based on the encoding scheme. In some implementations, corrected sequencing outputs or metadata values can be annotated or flagged for downstream quality control or audit processes.

In some implementations, the set of etches can be stored in a library, and the library can map a plurality of etches to corresponding metadata. For example, the library (e.g., library 114) can list etches 118 individually or in combinations along with corresponding contextual parameters (e.g., analyst identifier, sample collection timestamp, laboratory instrumentation identifier, etc.). In some examples, the library can include a lookup structure or encoded mapping table that defines relationships between individual molecular etches or combinations or molecular edges and respective contextual parameters (e.g., metadata values). In some examples, the mapping can be used to facilitate forward and reverse resolution of etches and corresponding metadata values (e.g., used by the encoding system 110 during etch selection and by the decoding system 140 during readout analysis).

In some implementations, the method 200 can include determining the one or more contextual parameters by comparing decoded values of the one or more etches to the corresponding metadata. For example, the decoding system (e.g., decoding system 140) can identify one or more read sequences 136 from the sequencing output 134 that match entries in the etch reference 144, and determine the associated contextual parameters (e.g., case number, analyst identifier, timestamp, etc.) based on the mapping defined in the library 114. In some implementations, determining the contextual parameters can include resolving combinations of read sequences 136 that correspond to compound metadata values. For example, the decoding system 140 can identify a set of etches corresponding to a full date value (e.g., separate read sequences encoding day, month, and year), and determine the date by resolving each combination based on the encoding scheme defined in the library 114. In some examples, the decoding system 140 can compare the determined contextual parameters to metadata values previously stored in association with the genetic sample 122 to confirm sample integrity or detect anomalies.

In some implementations, the method 200 can include using the one or more contextual parameters encoded by the one or more etches to verify an identity of the at least one genetic sample based on comparing the one or more contextual parameters and expected metadata. For example, the decoding system (e.g., decoding system 140) can determine contextual parameters based on read sequences 136 corresponding to etches identified in the sequencing output 134, and compare the determined contextual parameters to metadata values stored in a physical or digital record linked to the genetic sample 122 (e.g., a laboratory log, forensic chain-of-custody record, or LIMS entry). In some examples, verifying an identity of the genetic sample can include identifying or confirming that a set of detected metadata values (e.g., indicating an analyst identifier, case number, collection date, etc.) corresponds to or matches a set of stored metadata value recorded in a forensic or laboratory database in association with a sample identifier. That is, the contextual parameters can be used to verify an identity of the sample based on a combination of decoded metadata values, rather than encoding a direct sample identifier within the sample. For example, the decoding system 140 can compare a decoded analyst ID, collection timestamp, and instrument identifier to stored records and determine that the decoded combination uniquely matches a single entry corresponding the genetic sample in a case management system. For example, using combinations of contextual parameters can facilitate efficient and secure sample identification in workflows in which direct identifiers are excluded or obfuscated (e.g., for privacy or blinding), or can provide an additional layer of validation for provenance tracking or audit support.

In some implementations, the method 200 can include executing an error-detection code or an error-correction code on a sequencing output corresponding to the one or more etches to detect at least one error associated with interpretation of the one or more contextual parameters. For example, the decoding system (e.g., decoding system 140) can compare read sequences 136 associated with etches 120 against entries in the etch reference 144 to determine whether one or more of the read sequences includes a deviation from an expected read sequence. In some examples, the etches 118 can be configured to provide sequence-level redundancy (e.g., minimum edit distance constraints, dissimilarity metrics) to support detection of substitution, insertion, or deletion errors introduced during sequencing. In some examples, the decoding system 140 can apply an error-checking function (e.g., Hamming distance evaluation, etc.) to assess whether a noisy or truncated read sequence maps to a single etch entry within a predefined tolerance. For example, in response to execution of the error-detection or error-correction code, the decoding system 140 can associate the corresponding metadata value with the read sequence and optionally annotate the result with a confidence score. For example, in response to execution of the error-detection or error-correction code, the decoding system 140 can withhold or exclude the ambiguous read from downstream metadata interpretation. In some implementations, applying error detection or correction techniques can reduce the impact of sequencing artifacts and improve reliability of contextual metadata decoding (e.g., when etches are present at low input concentrations or near the sequencing error rate, etc.).

In some implementations, the method 200 can include flagging, responsive to multiplexed sequencing of a plurality of genetic samples comprising the at least one genetic sample, an unexpected metadata combination associated with one or more of the plurality of genetic samples. As used herein, multiplexed sequencing can refer to a technique or workflow used to sequence multiple samples (e.g., of a single organism, of multiple organisms, etc.) in combination (e.g., using a shared sequencing lane, cartridge, reaction environment, etc.) and in which individual samples are distinguishable based on combinations of sequencing reads and decoded contextual metadata. In some examples, flagging can include the decoding system 140 comparing contextual parameters decoded during a multiplexed sequencing run of a first sample and a second sample against expected metadata values stored in a physical or digital record associated with the first sample and/or the second sample. In some examples, an unexpected metadata combination can be detected based on a determination that a contextual parameter is duplicated, inconsistent, or missing across samples in the multiplexed run. In some examples, flagging can include the encoding system generating a structured indicator (e.g., flag), annotation, or alert corresponding with an unexpected metadata output. In some examples, the decoding system 140 can flag unexpected metadata combinations that fail to satisfy validation or error checks, including checks for sequencing depth, read count ratios, or known logical relationships between metadata fields.

In some implementations, the method 200 can include resolving the unexpected metadata combination using one or more resolution techniques. For example, resolution can include or refer to identifying, inferring, or recovering metadata associated with one or more genetic samples that have been flagged during multiplexed sequencing due to the presence of unexpected metadata combinations. In some examples, resolving can include applying automated logic, rule sets, or statistical models to determine valid metadata values for flagged samples. For example, the decoding system 140 can resolve an unexpected metadata combination by applying criteria such as read count thresholds, internal consistency rules, or comparative analysis with metadata values associated with other samples in the multiplexed sequencing run. In some implementations, resolving can include removing or excluding metadata values that are determined to be invalid, ambiguous, or inconsistent. In some examples, resolving can further include prompting for or receiving user input to adjudicate ambiguous metadata combinations, such as via a graphical user interface that presents flagged metadata for review. In some implementations, the decoding system 140 can annotate the sequencing output with resolution outcomes, confidence scores, or audit indicators based on the applied resolution technique. In some examples, resolution outcomes can be propagated to downstream workflows, such as quality assurance or recordkeeping systems, to reflect resolution of flagged metadata combinations.

In other examples, resolution can involve eliminating one or more flagged metadata values based on known inconsistencies or applying statistical models to infer the most probable combination. In some implementations, resolution can leverage prior records associated with the genetic sample, such as chain-of-custody data, lab processing logs, or LIMS metadata snapshots. In some cases, resolution can include disambiguating between conflicting values by tracing sample preparation steps linked to flagged metadata. In other examples, resolution can occur automatically by comparing detected metadata with a predefined set of valid configurations associated with the multiplexed run. Resolving flagged combinations can involve adjusting metadata assignments, excluding conflicting etches, or updating a digital record with adjudicated values. In some implementations, a graphical interface can present flagged combinations and recommended resolution actions to a laboratory technician for review. Resolution status can be appended to the genetic sample record and multiplexed run summary for downstream use. Resolution outcomes can feed into machine learning models to refine future flagging and resolution heuristics. In some examples, resolving the unexpected metadata combination can include storing a corrected version of the contextual parameters and noting the resolution method used (e.g., manual, heuristic-based, statistical inference) for future auditing. The system can track and report on the frequency and type of flagged and resolved issues across multiple multiplexed sequencing runs.

In some implementations, the method 200 can include validating the one or more contextual parameters by applying a threshold to sequencing read counts associated with each of the one or more etches, and a contextual parameter can be identified based on detecting a combination of etches with read counts satisfying the threshold. For example, the decoding system (e.g., decoding system 140) can quantify the number of read sequences 136 corresponding to the selected etches 120 identified in the sequencing output 134, and compare corresponding read counts to a predefined minimum threshold (e.g., absolute count, percentile cutoff for read counts, etc.). In some examples, a contextual parameter (e.g., sample collection date) can be identified if each read count of a predefined encoding group (e.g., for day, month, and year) meets or exceeds (e.g., satisfies) the read count threshold, thereby reducing the likelihood of inaccuracy due to background noise or sequencing error. In some implementations, the threshold can be dynamically adjusted based on total sequencing depth, the expected abundance of the etches 120, or known biases in the sequencing workflow. For example, threshold-based validation can be used in combination with error detection techniques described above to increase accuracy and confidence in contextual metadata interpretation during decoding.

Referring to FIG. 3, a block diagram depicting an implementation of a system 300 for data identification and data protection is shown, according to some implementations. System 300 can include client device(s) 310, metadata system(s) 330, data sources(s) 350, sample environment(s) 360, and sequencing system(s) 370. In various implementations, components of system 300 can communicate over network(s) 320. The client device(s) 310 can include one or more application(s) 312, library(s) 314, interface circuit(s) 316, and input/output circuit(s) 318. Further, the metadata system(s) 330 can include one or more processing circuit(s) 332, which can include one or more processor(s) 333 and memory(s) 334. The memory(s) 334 can include one or more content circuit(s) 335 and analysis circuit(s) 336. In some implementations, the metadata system(s) 330 can further include one or more database(s) 340. Although the various computing elements of FIG. 3 can be described in the singular form below (e.g., processor 333, database 340, etc.), it should be understood that the system 300 (e.g., a computing environment) can include two or more of any device/system described herein (e.g., two or more processors 333, databases 340, etc.).

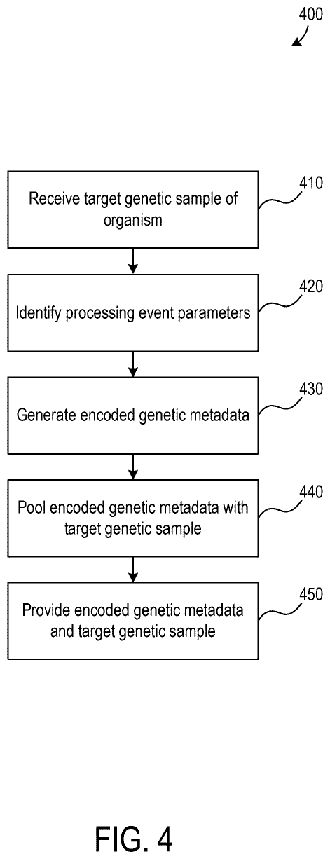

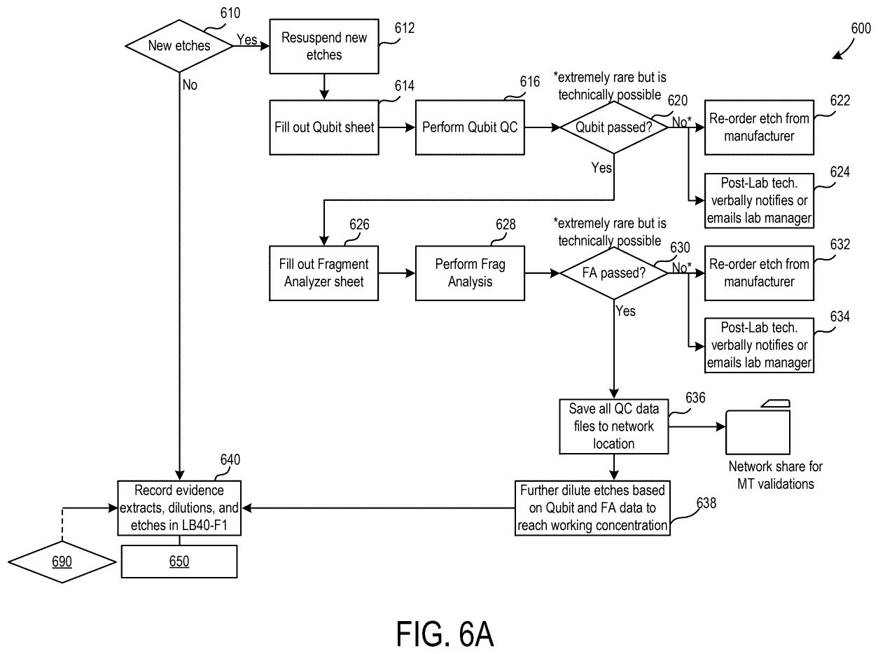

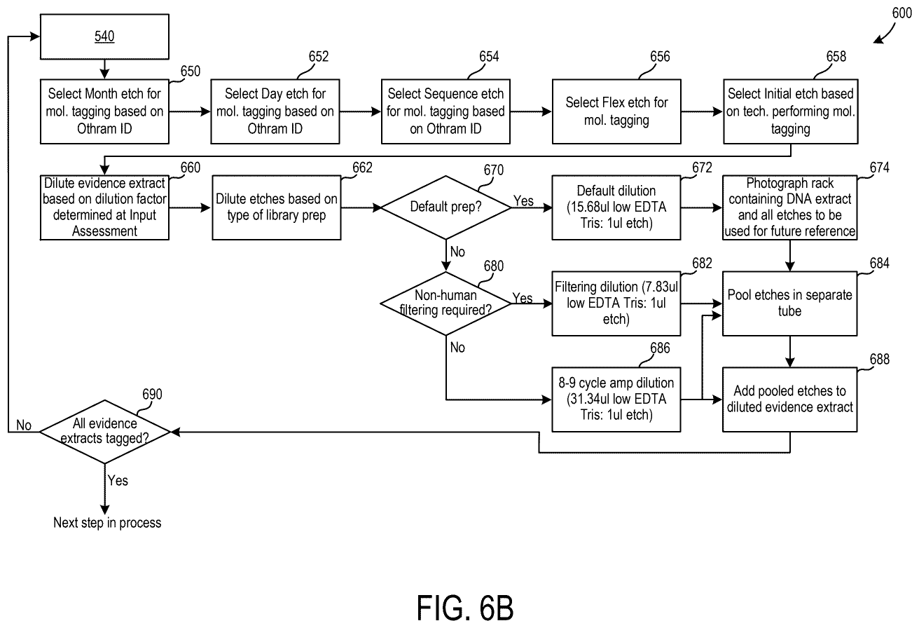

The various components of FIG. 3 can be implemented in a data identification or other metadata system to perform various the various computer-implemented methods, processes, and/or functionalities described herein (e.g., method 200 of FIG. 2, method 400 of FIG. 4, method 600 of FIG. 6). For example, the system 300 can include a receiving system (e.g., metadata system 330, analysis circuit 336, input/output circuit 318, etc.) for receiving a target genetic sample with capture event parameters and an identification system (e.g., metadata system 330, analysis circuit 336, etc.) to identify one or more processing event parameters for processing the sample (e.g., via the sequencing system 370). The system 300 can further include a generation system (e.g., metadata system 330, content circuit 335, analysis circuit 336, etc.) to generate encoded genetic metadata of the target genetic sample. For example, the generation system (e.g., analysis circuit 336) can generate the encoded genetic metadata (e.g., stored as one or more etches in a database accessible via network 320, such as database 340) by encoding data of at least one of the capture event parameters or at least one of the processing event parameters as the one or more etches of the encoded genetic metadata. The system 300 can further include a pooling system (e.g., sample environment 360, metadata system 330, analysis circuit 336, etc.) for pooling the one or more etches of the encoded genetic metadata with the target genetic sample. For example, the pooling system (e.g., analysis circuit 336, etc.) can be utilized to include and/or incorporate the one or more etches of the encoded genetic metadata with the target genetic sample in the sample environment (e.g., sample environment 360 or other genetic data store accessible via the network 320 or otherwise). The system 300 can further include a providing system (e.g., metadata system 330, analysis circuit 336, sample environment 360, etc.) for providing the encoded genetic metadata and target genetic sample (e.g., to the sequencing system 370 for sequencing, to another computing system or genetic system to perform PCR amplification, etc.). In some embodiments, the pooling system (e.g., analysis circuit 336) can maintain the target genetic sample in a processable or genetic readable format, as further described herein.

In some implementations, network 320 can include computer networks such as the Internet, local, wide, metro, or other area networks, intranets, satellite networks, other computer networks such as voice or data mobile phone communication networks, combinations thereof, or any other type of electronic communications network. Network 320 can include or constitute a display network. In various implementations, network 320 facilitates secure communication between components of system 300. As a non-limiting example, network 320 can implement transport layer security (TLS), secure sockets layer (SSL), hypertext transfer protocol secure (HTTPS), and/or any other secure communication protocol.

Client device 310 (sometimes referred to herein as a “client system”) can be a computing device, personal computer (PC), desktop computer, laptop computer, smartphone, tablet, smart watch, smart sensor, or any other device configured to facilitate receiving, displaying, and interacting with content (e.g., applications, etc.) or data (e.g., data corresponding to DNA, etches, etc.). Client device 310 can include an application 312 to receive and display content and to receive user interaction with the content. For example, application 312 can be a web browser. Additionally or alternatively, application 312 can be a mobile application. Client device 310 can also include an input/output circuit 318 for communicating data over network 320 (e.g., receive and transmit to metadata system 330).

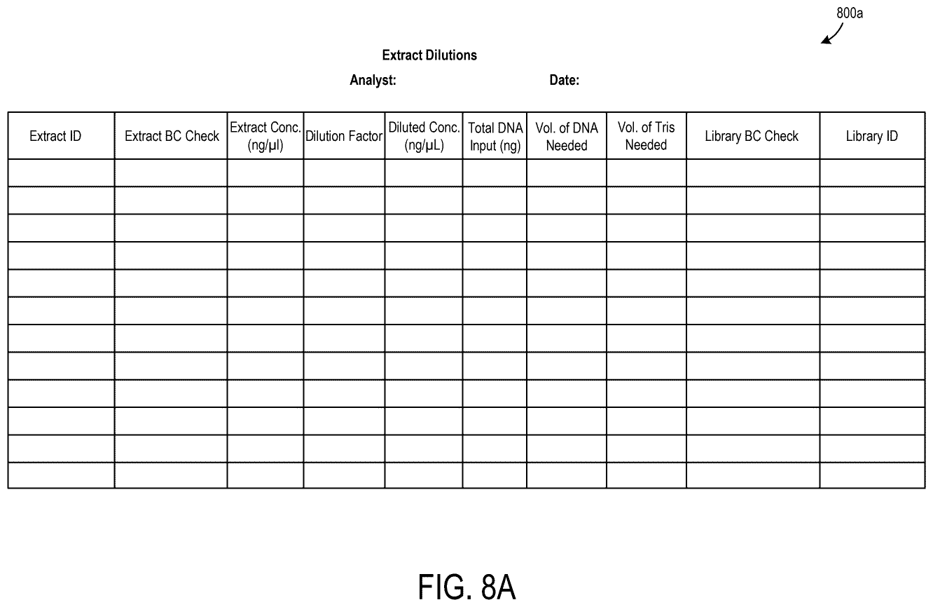

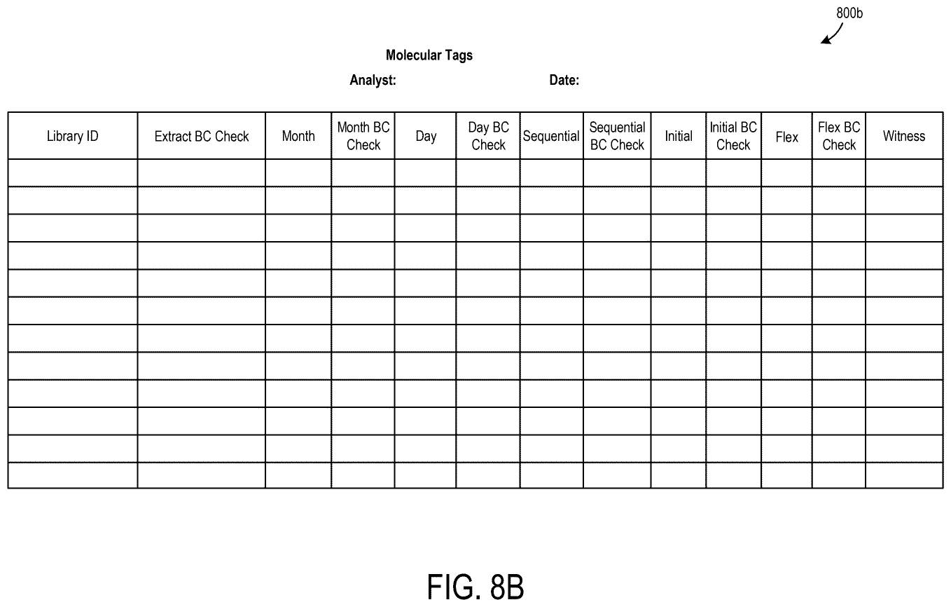

In general, the client device(s) 310 can execute a software application (such as application 312, e.g., a web browser, an installed application, or other application) to receive, access, and/or manage genetic data from other computing systems and devices over network 320. Such an application can be configured to receive or access genetic metadata from data sources 350 (e.g., via the sample environment 352). In one implementation, the client device 310 can execute a web browser application, which provides an interface (e.g., from content circuit 335) on a viewport of the client device 310. The client device 310 and application 312 can receive input from an input device (such as input/output circuit 318, e.g., a pointing device, a keyboard, a touch screen, or another form of input device). In response, one or more processors of the client device 310 executing the instructions from application 312 can request data from another device connected to the network 320 (e.g., the metadata system 330). The other device can then provide genetic data and/or other data to the client device 310, which causes the interface to be presented by the viewport of the client device 310 to a user for providing information or facilitating user interaction with the interface. In one implementation, application 312 can display a graphical user interface (GUI) such as an interactable web page and/or an interactive mobile application to a user (e.g., interfaces showing tabular data included on FIGS. 8A-8B, interfaces showing genetic metadata information or other genetic data, etc.).

Network 320 is composed of various network devices (nodes) communicatively linked to form one or more data communication paths between participating devices. The network 320 can facilitate communications between the various nodes, such as the client device 310, metadata system 330, data sources 350, and sequencing system 370 (e.g., using an OSI layer-4 transport protocol such as the User Datagram Protocol (UDP), the Transmission Control Protocol (TCP), Stream Control Transmission Protocol (SCTP), etc.). Each networked device includes at least one network interface for receiving and/or transmitting data, typically as one or more data packets. An illustrative network 320 is the Internet; however, other networks can be used. Network 320 can be an autonomous system (AS), i.e., a network that is operated under a consistent unified routing policy (or at least appears to from outside the AS network) and is generally managed by a single administrative entity (e.g., a system operator, administrator, or administrative group).

Application 312 is shown to include library 314 having an interface circuit 316. The library 314 can include a collection of software development tools contained in a package (e.g., software development kit (SDK), application programming interface (API), integrated development environment (IDE), debugger, etc.). For example, library 314 can include an application programming interface (API). In another example, library 314 can include a debugger. In yet another example, the library 314 can be an SDK that includes an API, a debugger, an IDE, and so on. In some implementations, library 314 includes one or more libraries having functions that interface with a particular system software (e.g., iOS, Android, Linux, etc.). Library 314 can facilitate embedding functionality in application 312. As a further example, library 314 can include a function configured to collect and report device analytics and a user can insert the function into the instructions of application 312 to cause the function to be called during specific actions of application 312 (e.g., during presentation of the genetic data as described in detail below). In some implementations, interface circuit 316 functionalities are provided by library 314.

Interface circuit 316 can be configured to provide one or more interfaces (e.g., a metadata interface for accessing, viewing, or updating genetic metadata of a target genetic sample). In various implementations, the interfaces can be presented on an application interface of application 312 presented in the viewport of client device 310. The interfaces provided by the interface circuit 316 can include various functionalities, such as providing a user genetic metadata (e.g., one or more DNA sequences or genetic material encoding information corresponding to a target genetic sample, etc.) for viewing; viewing deprotected (or anonymized) genetic data; reviewing output predictions, attributes of the genetic data, DNA markers, and DNA ranges, etc.

Interface circuit 316 can detect events within application 312. In various implementations, interface circuit 316 can be configured to trigger other functionality based on detecting specific events (e.g., generation of genetic metadata, updates to genetic data, etc.). In various implementations, library 314 includes a function that is embedded in application 312 to trigger interface circuit 316. It should be understood that events can include any action important to a user within an application and are not limited to the examples expressly contemplated herein.

The input/output circuit 318 is structured to send and receive communications over network 320 (e.g., with metadata system 330). The input/output circuit 318 is structured to exchange data, communications, instructions, etc. with an input/output component of the metadata system 330. In one implementation, the input/output circuit 318 includes communication circuitry for facilitating the exchange of data, values, messages, and the like between the input/output circuit 318 and the metadata system 330. In yet another implementation, the input/output circuit 318 includes machine-readable media for facilitating the exchange of information between the input/output device and the metadata system 330. In yet another embodiment, the input/output circuit 318 includes any combination of hardware components, communication circuitry, and machine-readable media.

In some implementations, the input/output circuit 318 includes suitable input/output ports and/or uses an interconnect bus (not shown) for interconnection with a local display (e.g., a touchscreen display) and/or keyboard/mouse devices (when applicable), or the like, serving as a local user interface for programming and/or data entry, retrieval, or other user interaction purposes. As such, the input/output circuit 318 can provide an interface for the user to interact with various applications (e.g., application 312) stored on the client device 310. For example, the input/output circuit 318 includes a keyboard, a keypad, a mouse, a joystick, a touch screen, a microphone, a haptic sensor, a car sensor, an IoT sensor, a biometric sensor, an accelerometer sensor, a virtual reality headset, smart glasses, smart headsets, and the like. As another example, input/output circuit 318, can include, but is not limited to, a television monitor, a computer monitor, a printer, a facsimile, a speaker, and so on.

Input/output circuit 318 can exchange and transmit data information (e.g., genetic data), via network 320, to all the devices described herein. In various implementations, input/output circuit 318 transmits data via network 320. Input/output circuit 318 can confirm the transmission of data. For example, input/output circuit 318 can transmit requests and/or information to metadata system 330 based on selecting one or more actionable items within the interfaces and dashboards described herein.

The metadata system 330 can include at least one logic device, such as a computing device having a processing circuit configured to execute instructions stored in a memory device to perform one or more operations described herein. The processing circuit can include a processor, such as a microprocessor, an application specific integrated circuit (ASIC), a field-programmable gate array (FPGA), etc., or combinations thereof. The processing circuit can include a memory, and the memory can include, but is not limited to, electronic, optical, magnetic, or any other storage or transmission device capable of providing processor(s) with program instructions. The instructions can include code from any suitable computer programming language such as, but not limited to, ActionScript®, C, C++, C#, HTML, Java®, JavaScript®, Perl®, Python®, Visual Basic®, and XML. In addition to the processing circuit, the metadata system 330 can include one or more databases (e.g., database 340) configured to store data. The metadata system 330 can also include an interface (e.g., content circuit 335) configured to receive data via the network 320 and to provide data from the content circuit 335 to any of the other systems and devices on the network 320.

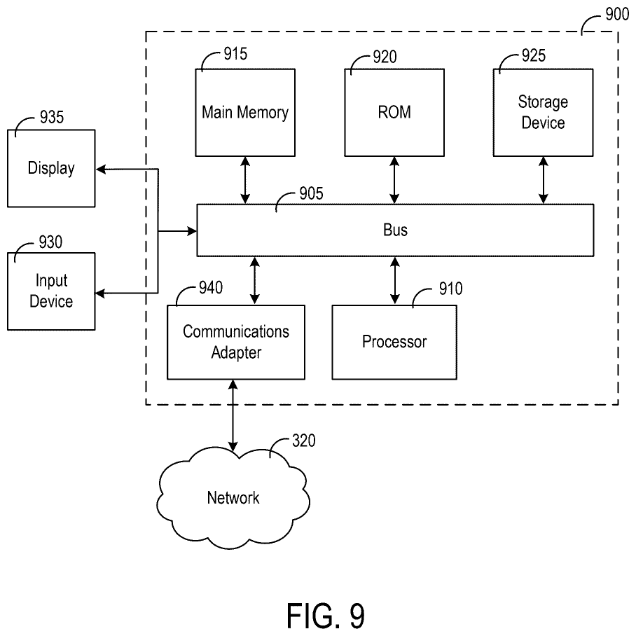

The metadata system 330 can be run or otherwise be executed on one or more processors of a computing device, such as those described below in detail with regard to FIG. 9. In broad overview, the metadata system 330 can include a processing circuit 332, a processor 333, memory 334, a content circuit 335, an analysis circuit 336, and a database 340. Data and/or interface(s) and dashboards generated by content circuit 335 can be provided to the client devices 310. The content circuit 335 can include a plurality of interfaces and properties, such as those described below in FIGS. 8A-8B.

The metadata system 330 can be a server, distributed processing cluster, cloud processing system, or any other computing device. Metadata system 330 can include or execute at least one computer program or at least one script. In some implementations, metadata system 330 includes combinations of software and hardware, such as one or more processors configured to execute one or more scripts.

As shown, metadata system 330 can include processing circuit 332, which can include processor 333 and memory 334. Memory 334 can have instructions stored thereon that, when executed by processor 333, cause processing circuit 332 to perform the various operations described herein. The operations described herein can be implemented using software, hardware, or a combination thereof. Processor 333 can include a microprocessor, ASIC, FPGA, etc., or combinations thereof. In many implementations, processor 333 can be a multi-core processor or an array of processors. Memory 334 can include, but is not limited to, electronic, optical, magnetic, or any other storage devices capable of providing processor 333 with program instructions. Memory 334 can include a floppy disk, CD-ROM, DVD, magnetic disk, memory chip, ROM, RAM, EEPROM, EPROM, flash memory, optical media, or any other suitable memory from which processor 333 can read instructions. The instructions can include code from any suitable computer programming language such as, but not limited to, C, C++, C#, Java, JavaScript, Perl, HTML, XML, Python and Visual Basic.

Metadata system 330 is shown to include database 340 and processing circuit 332. Database 340 can store data (e.g., received data such as sequenced DNA, stored data such as genetic metadata codes, etc.). For example, the database 340 can include data structures for storing information such as, but not limited to, genetic data (e.g., minor allele frequency (MAF) amount, a single nucleotide polymorphisms (SNPs) number (with genetic variation), a centimorgan (cM) length (e.g., either in aggregate or of the single longest shared segment), a Bit amount, a megabase (Mb) amount, DNA markers or DNA sequences, Chromosome position (inclusive), DNA ranges, DNA attributes, genetic protection model, relationship interfaces, relationship indices, subgroups, locations to physically stored generic data, crime scene data, other event data of the genetic samples, etc. The database 340 can be part of the metadata system 330, or a separate component that the metadata system 330 or the client device 310 can access via the network 320. The database 340 can also be distributed throughout system 300. For example, the database 340 can include multiple databases associated with the metadata system 330, the client device 310, or both. Database 340 can include one or more storage mediums. The storage mediums can include but are not limited to magnetic storage, optical storage, flash storage, and/or RAM. Metadata system 330 can implement or facilitate various APIs to perform database functions (i.e., managing data stored in database 340). The APIs can be but are not limited to SQL, ODBC, JDBC, NOSQL and/or any other data storage and manipulation API.

The data sources 350 can provide data to the various systems and computing devices of FIG. 3 (e.g., client device 310, metadata system 330, sequencing system 370, etc.). In some implementations, the data sources 350 can be structured to collect data from other devices on network 320 (e.g., client device 310) and relay the collected data to one or more of the various systems and computing devices of FIG. 3 (e.g., client device 310, metadata system 330, sequencing system 370, etc.). In one example, a user and/or entity can have a server and database (e.g., proxy, enterprise resource planning (ERP) system) that stores genetic data associated with an organism (e.g., an individual such as a human being, a canine or other non-human animal, a plant, etc.). In this example, the metadata system 330 can request data associated with specific genetic data stored in the data source (e.g., data sources 350) of the organism. Data sources 350 and other data processing or storage devices of FIG. 3 (e.g., database 340) can further transmit non-genetic data in addition to or alternatively from transmitting the genetic data.

The sample environment 360 can be a physical environment or a virtual environment for storing genetic metadata alongside genetic data. For example, sample environment 360 can be a physical environment such as a container (e.g., test tube, biological sample tube, etc.) or a virtual environment such as an electronic storage device or database (e.g., non-transitory memory accessible via network 320). For example, if the sample environment 360 is a physical device (e.g., sample tube), the sample environment 360 can be stored in a laboratory or testing facility (e.g., in a physical storage space or other repository configured to store multiple samples and/or sample environment(s) 360).

Adding DNA to the sample environment 360 in a physical context can include physically dispensing (e.g., using a pipette) one or more genetic sequences of the DNA material, which can be stored or suspended in a medium or other liquid included in sample environment 360. In a virtual context, the sample environment 360 can include data stored on electronic systems (e.g., in a database, such as database 340 of FIG. 3) or otherwise electronically stored (e.g., using a non-transitory CRM). For example, this electronic genetic data (e.g., data describing genetic data configured to be manipulated by a computer and/or sequencing system) can be structured and/or stored according to various structures, schemas, and/or formats (e.g., in tabular format, as shown on FIGS. 8A-8B). In some implementations, adding DNA to the sample environment 360 when the sample environment 360 is a virtual environment can include accessing one or more data storages via a network (e.g., network 320, as shown on FIG. 3) and further copying or storing electronic data corresponding to genetic data (e.g., copies of encoded genetic metadata, environmental data related to genetic processing conditions, encoding schemes, etc.). In some embodiments, one or more processing circuits can include a sample well or electro-fluid system (e.g., DNA input interface of a sequencing system, etc.). Furthermore, users and/or computing systems (e.g., via one or more processing circuits) can interact with the sample environment 360 by, for example, introducing synthetic DNA (e.g., genetic metadata) into the sample environment 360 without disturbing sample DNA contained therein, as further described below.

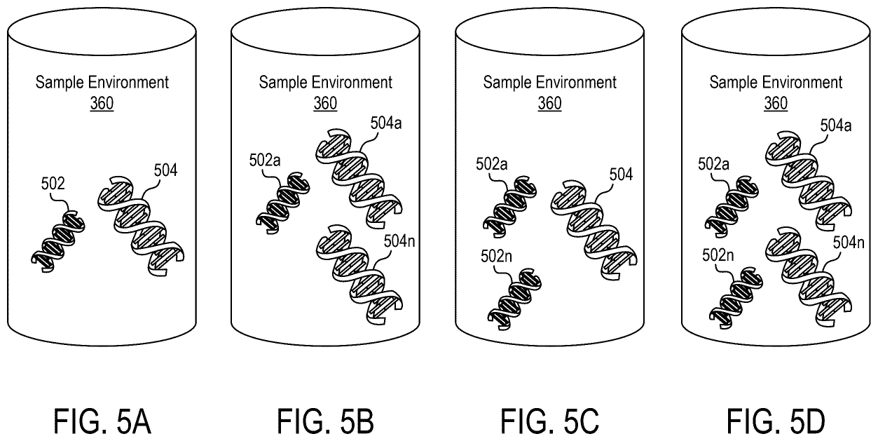

In some implementations, the sample environment 360 can be configured to store encoded genetic metadata of a target genetic sample and the target genetic sample for further processing or analysis (e.g., sequencing, polymerase chain reaction (PCR) processes, etc.). For example, the genetic metadata can be stored with the target genetic sample without modifying or attaching (e.g., chemically or covalently bonding) the metadata to the sample to preserve the DNA information of the sample in an unmodified or preserved state. In some implementations, the sample environment 360 can be used to provide the target genetic sample and encoded genetic metadata. Sample environment 360 is described in greater detail below regarding FIG. 5.

Referring to the metadata system 330, memory 334 includes content circuit 335. The content circuit 335 can be configured to generate content (e.g., data or information) for displaying to users or for integrating into various other systems and devices of FIG. 3 (e.g., data sources 350 and/or sample environment 360, sequencing system 370, etc.). For example, the content circuit 335 can generate an interface (e.g., GUI) that includes graphical representations of encoded metadata, one or more genetic samples, one or more predefined etches for encoding metadata, data structures such as tables including stored and/or generated metadata, data for verifying that metadata of a sample matches expected (e.g., stored data), etc. Additionally and/or alternatively, the content circuit 335 can provide structured or unstructured data decoupled from an interface, which can include data corresponding to encoded metadata, genetic samples, predefined etches for encoding metadata, stored and/or generated metadata, data for verifying that metadata of a sample matches, etc. The content circuit 335 is also structured to provide content (e.g., via a graphical user interface (GUI)) to the client device 310) and/or data (e.g., genetic data, metadata, etc.) over the network 320, for display within the resources or for further use by the computing devices of FIG. 3.

Memory 334 can also include analysis circuit 336. The analysis circuit 336 can be configured to receive, access, generate, and/or encode genetic data, including generating metadata describing genetic data stored in database 340, which can have been acquired as a result of identifying chromosomal sequences (e.g., sequences, a set of DNA markers, etc.) and determine genetic information (e.g., genetic sequences, species, identity, DNA portions or fragments, etc.). For example, the analysis circuit 336 can be configured to receive (e.g., via the network 320) genetic information of a sample (e.g., a hair sample, skin sample, or other biological sample or portion including genetic or DNA information of an organism) including parameters for capturing (e.g., collection parameters, capture parameters, parameters for collection of the sample, etc.) or processing the genetic information of the sample. The analysis circuit 336 can further generate encoded metadata of the sample by combining (e.g., encoding) one or more etches (e.g., stored in a library or database including predefined genetic metadata, such as database 340, data sources 350, and/or sample environment 360) corresponding to the parameters for capturing or processing. The analysis circuit 336 and/or other computing devices of FIG. 3 can further pool the etches of the metadata with the sample (e.g., using sample environment 352) and provide the metadata and sample (e.g., to the sequencing system 370).

In some implementations, the analysis circuit 336 can generate encoded genetic metadata of the target genetic sample. In some implementations, the encoded genetic metadata can include one or more etches (also referred to as synthetic genetic sequences, synthetic nucleic acid sequences, etc.). In some implementations, generating the encoded genetic metadata can include encoding data of at least one of the capture event parameters or at least one of the processing event parameters as the one or more etches of the encoded genetic metadata. For example, the etches of the encoded genetic metadata can be selected from a predefined library of synthesized non-organismal sequences and encode various information (e.g., sample collection data, environmental data, and/or any other data related to or associated with the target genetic sample, etc.). In another example, encoding can include combining one or more etches (e.g., base pair chains, etc.) such that each of the etches encodes a bit of information, and such that the bits encoded by the two or more etches can be combined according to an encoding scheme (e.g., a modified binary encoding scheme using unique nucleic acid sequences) to represent information related to the target genetic sample as genetic metadata.

In some implementations, the analysis circuit 336 can further pool the one or more etches of the encoded genetic metadata with the target genetic sample. For example, the analysis circuit 336 can include the one or more etches of the encoded genetic metadata with the target genetic sample in the sample environment. In some embodiments, the analysis circuit 336 can further provide the target genetic sample and encoded genetic metadata (e.g., for processing, sequencing, etc.). For example, the analysis circuit 336 can provide the sample environment 360 (or genetic material included in the sample environment 360) to a sample well of a sequencing system (e.g., for genetic identification or analysis of the encoded metadata and/or of the target genetic material). In some embodiments, pooling maintains the target sample in a processable or genetic-readable format. For example, genetic metadata can be added to the environment unbonded (e.g., separately, separate, distinct from, etc.) from the sample DNA, and the genetic metadata can include one or more sequences that do not interfere with processing of the sample (e.g., that do not map or align with a reference genome of the organism providing the sample). For example, a reference genome of the organism can include a digital nucleic acid sequence (e.g., Human Reference Genome data stored in a database, such as database 340) assembled as a representative example of a species' genetic composition (e.g., DNA) and used for mapping and aligning sample DNA fragments (e.g., for sequencing, for genetic identification, etc.). As used herein, a reference genome can include a synthetic reference genome. In some implementations, a synthetic reference genome can include a reference model (e.g., genetic dataset) containing genetic sequences that do not conflict (e.g., are distinct from) a standard organismal reference genome (e.g., Human Reference Genome (GRCh38/hg38)). For example, a synthetic reference genome can be developed and configured to be utilized by a sequencing system to provide DNA reads that do not conflict with a Human Reference Genome. Such synthetic reference genome can be developed using various techniques, including modifying a standard reference genome used for sequencing (e.g., Human Reference Genome (GRCh38/hg38) for sample DNA of human organism(s), GRCm39 or mm39 for laboratory mice sample DNA, etc.) to provide reads for metadata that do not conflict with the Human Reference Genome. As another example, the synthetic reference genome can include a modified Human Reference Genome configured to provide reads (e.g., genetic information, mapped genetic information, etc.) from non-human DNA fragments (e.g., from metadata sequences) in addition to reads from human (or human-like) DNA fragments (e.g., from target sample sequences). In some implementations, a reference genome can include a group of mapped chromosomes (or digitalized dataset) aligning to DNA of one or more organisms, and a synthetic reference genome can be an additional chromosome (or dataset) aligning to etch DNA (e.g., metadata).

Sequencing system 370 can include a personal computer, laptop/tablet, and/or other electronic computing devices configured to determine genetic information of inputted genetic data (e.g., the identity of the organism corresponding to the sample, etc.). The sequencing system 370 can interact with one or more of the components of FIG. 3 (e.g., sample environment 352, input/output circuit 318, etc.) to analyze etches included with a genetic sample of an organism. For example, the sequencing system 370 can receive or access genetic data included in the sample environment 360 or the data sources 350 for processing and/or sequencing. In some implementations, the sequencing system 370 can be configured to perform PCR techniques (e.g., by an operator or user, or by the sequencing system 370 via an onboard processor, etc.) to amplify genetic information before sequencing. In some implementations, the sequencing system 370 can receive a target genetic sample and encoded genetic metadata (e.g., after extraction using the sample environment 360, etc.) for sequencing. The sequencing system 370 can receive the sample genetic material using a physical and/or electronic interface (e.g., sample well or sample holder configured to receive genetic material for processing). The sequencing system 370 can implement one or more sequencing methodologies or processes, which can include Sanger Sequencing (e.g., for precise, short-sequence reads), next-generation sequencing (NGS) (e.g., for accurate sequencing of voluminous or extensive genetic data), and/or additional or alternative sequencing or genetic data processes. In some implementations, the sequencing system 370 can sequence a sample DNA portion and corresponding genetic metadata to determine genetic information related to the sample DNA portion (e.g., based on sequencing the included metadata). The sequencing system 370 can sequence the genetic metadata separately from the sample genetic information and output one or more sequences corresponding to the genetic metadata and to the sample genetic information, respectively.

Referring now to FIG. 4, a flowchart for a method for generating and incorporating encoded genetic metadata with a target genetic sample of an organism is shown, according to some implementations. One or more systems, sub-systems, or components described herein (e.g., system 100 of FIG. 1, metadata system 330 of FIG. 3, etc.) can be configured to perform method 400. In some the steps or blocks of method 400 can be executed sequentially or in parallel (e.g., block 420 and 430 can be performed in parallel).

In broad overview of method 400, at block 410, the one or more processing circuits (e.g., metadata system 330 in FIG. 3) can receive a target genetic sample of the organism. At block 420, the one or more processing circuits can identify one or more processing event parameters. At block 430, the one or more processing circuits can generate encoded genetic metadata of the target genetic sample. At block 440, the one or more processing circuits can pool the encoded genetic metadata with the target genetic sample. At block 450, the one or more processing circuits can provide the target genetic sample and encoded genetic metadata. Additional, fewer, or different operations can be performed depending on the particular arrangement. In some implementations, some, or all operations of method 400 can be performed by one or more processors executing on one or more computing devices, systems, or servers. In various implementations, each operation can be re-ordered, added, removed, or repeated.

Referring to method 400 in more detail, at block 410, the one or more processing circuits (e.g., metadata system 330 in FIG. 3) can receive the target genetic sample of the organism corresponding with capture event parameters. In some implementations, the capture event parameters can include one or more of a crime scene location, an officer identity, a suspect identity, a victim identity, a time, a timestamp of capture, a crime scene parameter, a unique reference number, a case number, a chain of custody, a crime type, and witness statements. The processing event parameters can include collection parameters related to the collection of a DNA sample and/or describing the circumstances of sample's collection and/or capture. The target genetic sample can include genetic data or genetic sequences including, but not limited to, genetic data related to the inherited or acquired genetic characteristics of a person (or animal) or an individual, deoxyribonucleic acid (DNA), ribonucleic acid (RNA), DNA marker genotypes (e.g., specific location of a gene or other DNA sequence on a chromosome), genetic sequences, historical diseases, disease risk, traits, ancestry, etc. In some implementations, the one or more processing circuits can receive multiple (e.g., at least two) genetic datasets or samples of multiple people (e.g., at least two) that can be analyzed as described below regarding the target genetic sample. In some implementations, the target genetic sample of the organism includes DNA of a human organism (e.g., a homosapien) or DNA of a non-human organism (e.g., canine, feline, other animal, plant, etc.).

At block 420, the one or more processing circuits can identify one or more processing event parameters. In some implementations, at block 420, the one or more processing circuits can identify (e.g., assign, determine, receive, etc.) one or more processing event parameters for processing the target genetic sample. Processing can include sequencing (e.g., using NGS sequencing techniques and/or systems), amplification or other preparation processes performed before sequencing (e.g., PCR), and/or any other data identification or data processing operation using the target genetic sample and/or using encoded genetic metadata. In some implementations, the one or processing event parameters includes one or more of processing instructions, sequencing options, testing protocols, preservatives, unique reference numbers, case numbers, operator identity, training records, processing locations, times, timestamps of processing, quality assurance checks, compliance standards, calibration data, equipment use data, consumable or reagent tracking data, processing results, error logs, and/or data integrity measures.

At block 430, the one or more processing circuits can generate encoded genetic metadata of the target genetic sample. In some implementations, at block 430, the one or more processing circuits can generate the encoded genetic metadata including one or more etches (e.g., synthetic oligonucleotides, molecular tags, etc.). Further, generating the encoded genetic metadata at block 430 can include encoding data of at least one of the capture event parameters (e.g., collection data) or at least one of the processing event parameters (e.g., timestamp, operator/technician identifier, etc.) as the one or more etches of the encoded genetic metadata. In some implementations, the one or more etches of the encoded genetic metadata include at least one non-human sequence. For example, the one or more etches of the encoded genetic metadata can diverge from a reference genome of the organism (e.g., the organism providing the sample) such that the encoded genetic metadata is not interpreted as DNA of the organism during sequencing. For example, the DNA of the organism can be non-interfering data such that the organismal DNA can be sequenced separately (e.g., provide genetic reads or mapped genetic fragments distinct from genetic reads or mapped genetic fragments determined from sequencing target sample DNA). In some implementations, the one or more etches of the encoded genetic metadata diverge from a reference genome by diverging from a standard reference genome (e.g., a typical or unmodified genome commonly used in sequencing, so as the Human Reference Genome) and aligning with a modified reference genome (e.g., synthetic reference genome used in sequencing and configured to align with both metadata DNA fragments and sample DNA fragments).

In some implementations, generating the encoded genetic metadata at block 430 includes synthesizing two or more etches diverging from a human reference genome. For example, at block 430, encoded genetic material (e.g., metadata) used to represent information about sample genetic material can be received (e.g., from a manufacturer after synthesis), selected (e.g., from a preexisting library or other data store), or otherwise generated using one or more of using various DNA fragment or genetic sequence synthesis techniques. In some implementations, generating the encoded metadata at block 430 further includes combining at least two of the two or more etches to represent at least one of the capture event parameters or processing event parameters as at least one non-human genetic sequence. For example, combining can include pooling one or more etches to generate encoded metadata indicative of a sample.

In some implementations, generating the encoded genetic metadata at block 430 includes processing or pre-processing the encoded genetic metadata for further analysis. Prior to sequencing, for example, one or more processing circuits and/or an operator can generate one or more reagents for amplifying the genetic material metadata. In some implementations, generating the reagents can include synthesizing one or more genetic primers (e.g., synthetic DNA strands) to selectively bind (e.g., via covalent bonds) to at least one of the etches of the genetic material metadata (e.g., to a target DNA molecule). Further, the processing circuit(s) and/or operator can apply the reagent to the genetic metadata material via a sample environment (e.g., within a test tube or other container configured to be received by a sample well or sample holder of a sequencing system).

In some implementations, generating the encoded genetic metadata at block 430 includes implementing an encoding scheme. For example, a predefined methodology or approach (e.g., encoding scheme or technique) can be selected for encoding genetic etches as metadata, and the metadata can be generated and interpreted based on the selected encoding scheme. In some examples, the encoding scheme includes one or more from the group consisting of: Manchester encoding, Differential encoding, Non-Return-to-Zero Inverted (NRZI) encoding, Pulse-code modulation (PCM), Binary Phase Shift Keying (BPSK), Miller encoding, and 8b/10b encoding. A variety of other encoding techniques or frameworks can be used in various implementations for encoding genetic data (e.g., etches) as metadata.

At block 440, the one or more processing circuits can pool the encoded genetic metadata with the target genetic sample. In some implementations, at block 440, the one or more processing circuits can pool the one or more etches by including the one or more etches of the encoded genetic metadata in a sample environment. Further, pooling can maintain the target genetic sample in a processable or genetic-readable format (e.g., capable of successful sequencing without interference from included metadata).

In some implementations, pooling at block 440 can include adding the target genetic sample of the organism to a sample environment including a container for storing genetic material. For example, the sample container can include a test tube or other container for storing, preserving, and/or preparing genetic material (e.g., for sequencing). In some implementations, the target genetic sample can be included with the sample environment before block 440 is reached, in which case the method 400 can include bypassing adding the sample to the sample environment and further performing the steps described below.

In some implementations, pooling at block 440 can further include adding one or more etches of the encoded genetic metadata to the sample environment by including the one or more etches of the encoded genetic metadata in the sample environment unbonded with the target genetic sample. In some implementations, the one or more sequences of the encoded genetic metadata can be added to a separate container (e.g., for dilution, for preparation, or for various other purposes) before pooling the encoded genetic metadata in the sample container with the target genetic sample. In other implementations, each of the etches/sequences used to encode metadata can be directly added to the sample environment including the target genetic sample. As further described herein, the sample environment can be a physical or virtual environment, and can be configured to be received by a genetic processing and/or sequencing system (e.g., using a sample well of the sequencing system to receive genetic material from or extracted from the sample environment, by the sample well of the sequencing system being the sample environment, etc.). The sample environment is described in greater detail with respect to FIG. 5.

In some implementations, adding the one or more etches of the encoded genetic metadata to the sample environment further includes adding the one or more etches of the encoded genetic metadata to a metadata sample environment. In some embodiments, the metadata sample environment can incorporate the same or similar features as described the sample environment 360 of FIGS. 3 and 5. For example, the metadata sample environment can be a physical container (e.g., test tube, sample well) for storing and/or pooling the one or more etches of the encoded genetic metadata from the metadata sample environment before the encoded genetic material is added to the sample environment. In some implementations, adding the target genetic sample to the sample environment further includes adding the one or more etches of the encoded genetic metadata separately from the one or more genetic sequences of the target genetic sample. For example, etches of the encoded genetic metadata and genetic sequences of the target genetic sample can be stored in the same physical container (e.g., suspended in the same medium), and each can remain unbonded with other contents (e.g., sequences) included in the container.

In some implementations, pooling at block 440 can further include providing the sample environment including the one or more etches of the encoded genetic metadata and the target genetic sample to a sequencing system. For example, a laboratory analyst or other user can provide a physical container including the sample data and metadata and/or the genetic contents (e.g., sequences) contained therein to an input interface of a sequencing system (e.g., sample well). In other implementations, one or more processing circuits can be used (e.g., in conjunction with a mechanical, electromechanical, fluid-based (e.g., hydraulic), or electro-fluid system or apparatus) to automatically provide the encoded genetic metadata to a processing or sequencing system, as described in greater detail regarding FIG. 5 (e.g., regarding sample environment 360).

At block 450, the one or more processing circuits can provide the target genetic sample and encoded genetic metadata. In some implementations, at block 450, providing the target genetic sample can include adding the target genetic sample to a sample environment and adding the encoded genetic metadata to the sample environment unbonded with the target genetic sample. Block 450 can further include providing the sample environment including the one or more etches of the encoded genetic metadata and the target genetic sample to a sequencing system (e.g., to a sequencing machine, to a sample well of a sequencing apparatus, etc.). In some implementations, providing the target genetic material and target genetic sample at block 450 includes providing the target genetic sample and/or encoded genetic metadata to an evidentiary system (e.g., to a criminal justice entity, to a judge in a courtroom proceeding, via a graphical user interface available on the Internet, etc.) or to a health or healthcare system (e.g., to a hospital, to a computing system of an medical facility, to a genetics laboratory, etc.).

In some implementations, the method 400 further includes sequencing the pooled contents of the sample environment that includes the one or more etches of the encoded genetic metadata and the target genetic sample. For example, one or more processing circuits (e.g., sequencing system 370 of FIG. 3) can receive the sample environment including the sample and metadata and further process the genetic material contained therein (e.g., at block 450 or otherwise). In other implementations, the method 400 further includes processing (e.g., amplifying using PCR, etc.) the genetic data before providing the sample environment to a sequencing device for sequencing. For example, processing event parameters (e.g., data) used for processing, amplification, and/or sequencing can be identified and encoded using the genetic metadata, as described above. As used herein, “processing” can include any genetic identification, genetic preparation, or other genetic process (e.g., genomic modification/editing, etc.), and “sequencing” can include any process to identify genetic sequences and/or determine a genetic profile of an organism using sample DNA.

In some implementations, the method 400 further includes analyzing, by the one or more processing circuits, the portion of the genetic dataset and genetic material metadata using the one or more processing event parameters. For example, the target genetic sample (e.g., hair sample of a suspect, skin sample of a victim, etc.) and encoded genetic metadata (e.g., oligonucleotide pairs unbonded from the sample) can be received and sequenced by a sequencing system (e.g., sequencing system 370, as shown on FIG. 3) to determine genetic information corresponding to the sample and/or the metadata. In some implementations, receiving can include the one or more processing circuits (e.g., sequencing system) accessing or identifying genetic material deposited in a sample well of the sequencing system, or otherwise accessible to the sequencing system (e.g., using a standalone extraction system).

In some implementations, the method 400 can further include sequencing, by the one or more processing circuits, the target genetic sample and encoded genetic metadata using a synthetic reference genome. For example, sequencing can include performing genetic processing (e.g., using a computing or processing device) and/or analysis to determine strings of bonded base pairs (e.g., etches, sequences, DNA profiles, etc.) aligned with a reference genome. In some implementations, the synthetic reference genome (e.g., etch reference) can include a digital genetic sequence, and the digital genetic sequence can align or map with the one or more etches of the encoded genetic metadata. For example, a Human Reference Genome dataset and a synthetic reference genome dataset can be used by a sequencing machine to provide reads (e.g., sequences aligned to portions of the Human Reference Genome and portions of the synthetic reference genome) mapped to both encoded genetic metadata and target genetic sample DNA.

In some implementations, the method 400 further includes comparing, by the one or more processing circuits, the one or more etches of the sequenced encoded genetic metadata to a genetic reference dataset. For example, a genetic reference dataset can include a sequenced or mapped human genome, sequenced/mapped genomes, and/or other information used in sequencing genetic material of an organism. For example, a reference genome of the organism can include a digital nucleic acid sequence dataset assembled as a representative example of a species' genetic composition (e.g., DNA) and used for mapping and aligning sample DNA fragments (e.g., for sequencing, for genetic identification, etc.). For example, the genetic reference dataset can include a synthetic reference genome dataset including modifications to a standard reference genome used for sequencing (e.g., a modified version of a dataset of the Human Reference Genome (GRCh38/hg38)). For example, the synthetic reference genome can include a modified Human Reference Genome configured to provide reads or other genetic information (e.g., mapped sequences, encoded information included in genetic metadata, etc.) from non-human DNA fragments (e.g., from metadata sequences) in addition to reads from human DNA fragments (e.g., from target sample sequences). In this example, the modified synthetic reference genome dataset can incorporate metadata sequences alongside or with common human DNA sequences. That is, during genetic analysis, systems can obtain reads or other genetic information from the target human DNA fragment and from associated metadata sequences. As shown, the dataset can be enriched with additional contextual information for improved analysis and recordation. Further, comparing can include determining at least one of the capture event parameters or processing event parameters encoded in the genetic material metadata (e.g., determining a timestamp represented by genetic metadata, determining a collection data represented by the genetic metadata, etc.). For example, determining the capture event parameter(s) and/or processing event parameter(s) can include executing a data processing algorithm to determine data of capture, collection, or processing corresponding to the target genetic sample and encoded using the encoded genetic metadata (e.g., an encoded timestamp of collection of a sample).

In some implementations, the method 400 further includes generating one or more reagents for amplifying the genetic material metadata. For example, the method 400 can include generating various compositions (e.g., reagents chemicals, molecules, genetic material, etc.) that can interact with DNA or other genetic material and cause various reactions (e.g., formation of molecular bonds between genetic material, amplification of etches or genetic sequences for processing/sequencing, etc.) when exposed to the DNA/genetic material. In implementations involving polymerase chain reaction (PCR) amplification, such compositions/reagents can include combinations of DNA polymerase, genetic fragments/primers, genetic probes, nucleotides (dNTPs) (e.g., composition of deoxyadenosine triphosphate (dATP), deoxycytidine triphosphate (dCTP), deoxyguanosine triphosphate (dGTP), and deoxythymidine triphosphate (dTTP)), buffers (e.g., compositions to provide icon and pH environment for amplification), cofactors and/or stabilization chemicals (e.g., MgCl2 (Magnesium Chloride), KCl (Potassium Chloride), etc.), and/or various other additives (e.g., Betaine and DMSO (Dimethyl Sulfoxide), etc.). Such reagents can be pre-synthesized and/or purchased from a third-party, or can be generated each time the reagent is used (e.g., for amplification, being generated after receipt of genetic material and by using data of the genetic material to generate the reagent(s)). For example, generating the reagents can include synthesizing one or more genetic primers or probes to selectively bind to at least one of the one or more etches of the genetic material metadata. In some implementations, generating the reagents further includes applying (e.g., manually by an operator, automatically through an electro-fluid system, etc.) the one or more reagents to the genetic material metadata via the sample environment. For example, an operator or technician can apply the reagents to a container (e.g., sample container, sample storage environment, test tube, etc.) including genetic material metadata using a physical device (e.g., dropper, pipette, etc.) for the genetic material metadata to be accurately sequenced.

In some implementations, the method 400 further includes receiving data corresponding to the generation of the encoded metadata. For example, the one or more processing circuits can receive, identify, or access data from various data sources (e.g., one or more databases and/or data stores, such as database 340 and/or data sources 350 of FIG. 3) connected through a network (e.g., network 320). The data corresponding to the generation of the encoded metadata can include copies of data stored/encoded in the metadata (e.g., a copy of an encoded timestamp used for later verification, etc.) and/or any other data related to a genetic sample and/or genetic metadata of the sample (e.g., generation data, update data, access data, etc.). In some implementations, the method 400 further includes storing data corresponding to the generation of encoded genetic metadata in an electronic storage environment. For example, an electronic storage environment can be a database (e.g., database 340 of FIG. 3), computer-readable storage medium (CRM), and/or any other electronic means of storing electronic data or genetic data.

In some implementations, the method 400 further includes determining an update to the genetic metadata. For example, the one or more processing circuits can determine, based on stored data corresponding to the generation of encoded genetic metadata, an update to the genetic metadata based on comparing data including the encoded genetic metadata to new data. An update to the genetic metadata can include a change, alteration, addition to, deletion from, and/or any other modification or rearrangement of data corresponding to the genetic metadata or to a sample (e.g., DNA sample) corresponding to the genetic metadata. For example, an update can include a chemical and/or molecular change to the metadata itself (e.g., the addition of new DNA portion to encode new data, removal of previously-used DNA portion, etc.), a change to stored data corresponding to the genetic metadata and/or sample genetic material (e.g., a change to a database containing copies of data included in the metadata for verification), and/or any other modification (e.g., laboratory implementation of new genetic identification scheme, legal name change of a technician whose initials are encoded as an “analyst” in the metadata, later-added corresponding court filing related to the genetic sample described by the genetic metadata, etc.). Comparing data can include the one or more processing circuits evaluating and identifying a difference between metadata and new data (e.g., determining that a timestamp encoded in genetic metadata diverges from a newly received and/or verified timestamp, etc.).

In some implementations, the method 400 further includes generating updated encoded genetic metadata. For example, the updated encoded genetic metadata can be generated as described above regarding original encoded genetic metadata (e.g., by combining two or more etches, by synthesizing a DNA portion, etc.). For example, generating the updated encoded genetic metadata can further include combining (e.g., by the one or more processing circuits or a human operator) two or more etches stored in a predefined library (e.g., in a physical collection of DNA fragments/etches, in a digital library with stored data digitally representing etches and/or portions, etc.) to represent the new data (e.g., via an encoding scheme using combinations of one or more DNA fragments to encode data). In some implementations, the method 400 further includes updating stored data to represent the new data. For example, a database can include information stored in encoded genetic metadata (e.g., include digital copies of timestamps, identifiers, dates, technician initials, etc. included in the metadata), and this information can be updated in response to the generation of updated metadata to represent (e.g., serve as a copy of) the new/updated data. In some implementations, the method 400 further includes comparing the one or more etches of the sequenced encoded genetic metadata to a genetic reference dataset to determine at least one of the capture event parameters or processing event parameters encoded in the genetic material metadata. The method 400 can further include determining that the determined at least one capture event parameter or processing event parameter aligns with at least one expected (e.g., stored, verified, etc.) capture event parameter or expected processing event parameter.

In some implementations, the encoded genetic metadata does not interfere with sequencing of the target genetic sample. That is, method 400 maintains the target genetic sample in a processable or genetic-readable format such that either or both of the target genetic sample and encoded genetic metadata can be accurately amplified, sequenced, or otherwise processed after encoded genetic metadata is added to a sample environment containing the target genetic sample. Including non-interfering metadata can avoid the metadata aligning or mapping to a reference genome used in sequencing the sample (e.g., in embodiments using standard reference genomes, such as an unmodified Human Reference Genome), and can further avoid sequencing or other genetic processing errors or interference resulting from the introduction of encoded genetic metadata to the sample genetic material. In other embodiments (e.g., in embodiments using a modified or synthetic reference genome configured to align with non-human metadata sequences), the one or more etches of the encoded genetic metadata can be sequenced in the same batch as the target genetic sample (e.g., simultaneously, one immediately after another, etc.) but provide separate non-interfering reads (e.g., by the metadata sequences being aligned with known non-organismal sequences of a synthetic reference genome and by organismal or sample sequences being aligned with standard organismal sequences included in both a reference genome and a modified synesthetic reference genome). Thus, including encoded genetic metadata that does not align to a reference genome of the organism providing the sample (e.g., non-human DNA metadata for a sample of a human, etc.) provides technical benefits by improving the accuracy and efficiency of genetic identification processes and improving the efficiency of computing systems used for sequencing and/or other genetic identification. While it is understood that the genetic sample herein can be genetically sequenced after metadata is added to the sample environment, it should be understood that adding encoded metadata to the sample environment with the target genetic sample according to method 400 further preserves the target genetic sample for processing in accordance with various additional and/or alternative genetic processes, such as pre-sequencing amplification (e.g., PCR), reference genome creation and/or modification, DNA cloning, DNA modification/editing, etc.

In some implementations, the method 400 further includes receiving sequenced encoded genetic metadata from sequencing the target genetic sample and encoded genetic metadata. For example, one or more reads or mapped genetic sequences corresponding to the encoded genetic metadata can be output by a sequencing system after sequencing the sample environment with the target genetic sample and encoded genetic metadata. In some implementations, in response to receiving the sequenced encoded genetic metadata, the one or more processing circuits can determine at least one error corresponding to the encoded genetic metadata by executing an error-detection code or an error-correction code, wherein the error-correction code analyzes the sequenced encoded genetic metadata to identify and output the at least one error. For example, the error-correction code (e.g., error correcting code, ECC, etc.) or error-detection code can include one or more from a group consisting of: Hamming codes, Reed-Solomon codes, Low-Density Parity-Check (LDPC) codes, turbo codes, Cyclic Redundancy Check (CRC) codes, Bose-Chaudhuri-Hocquenghem (BCH) codes, RS232, Ethernet, TCP, UDP, Golay codes, Goppa codes, Viterbi decoders, multidimensional parity codes, checksum codes, hash codes, message authentication codes, alternant codes, AN codes, Berger codes, forward error correction codes, generalized minimum-distance codes, rank error-correction codes, and remote error indication codes. While examples are provided above, various additional and/or alternative error-detection and/or error-correction codes can be implemented in various embodiments. In some implementations, the at least one error can be reported (e.g., provided, sent to, etc.) to an entity or user (e.g., in response to detection).

Generally, as shown in method 400, a physical or virtual environment can be used as a sample environment for including encoded genetic metadata with a DNA sample. As described above regarding sample environment 360 and FIGS. 3-5, in a physical context, the sample environment can include a test tube or other physical container (e.g., stored in a laboratory, etc.), and in a virtual context, the sample environment can include a database or other electronic storage medium (e.g., computing system, processing system, CRM, solid-state drive, etc.). Further, as described above, the sample environment 360 can be an electro-fluid system or interface configured to receive, manipulate, and/or process physical genetic material (e.g., DNA sequences) electronically (e.g., by one or more processors executing instructions stored using one or more non-transitory computer-readable storage media, by using a microchip with multiple wells configured to storage DNA fragments for analysis, etc.). In embodiments where the sample environment 360 is a purely physical environment (e.g., a test tube), the method 400 can include one or more users (e.g., laboratory technicians, operators, etc.) performing one or more of the steps including in method 400 in addition to, in combination with, or instead of one or more processing circuits performing such steps. For example, pooling encoded genetic metadata with a target genetic sample can include a technician physically interacting with the sample environment 360 by using a physical dispenser (e.g., dropper, pipette, etc.) to combine and/or dilute the metadata and/or the sample. In embodiments involving a virtual (e.g., electronic) environment, electro-fluid-based environment, or other electronic sample environment 360, one or more processing circuits (e.g., of a sequencing machine executed by one or more electronic components to control a sample well, etc.) can perform the various steps included above (e.g., electronic signals can cause the one or more processing circuits to mix solutions containing metadata with solutions containing the sample via the sample well, etc.).

Referring now to FIGS. 5A-5D, block diagrams depicting an example of sample environment 360 for data identification and data protection is shown, according to some implementations. As described regarding FIG. 3, the sample environment 360 can be a physical environment or a virtual environment for storing genetic metadata alongside genetic data, such as a container (e.g., test tube, biological sample tube, etc.) or an electronic storage device or database (e.g., non-transitory memory accessible via network 320). In some implementations, the sample environment 360 can be configured to store encoded genetic metadata of a target genetic sample and the target genetic sample for further processing or analysis (e.g., sequencing, polymerase chain reaction (PCR) or other amplification processes, etc.) without modifying or attaching (e.g., chemically or covalently bonding) the metadata to the sample (e.g., to preserve the DNA information of the sample in an untouched form).

Referring now to FIG. 5A, the sample environment 360 can include a composition including metadata 502 and sample data 504. As shown, in some implementations, the metadata 502 and sample data 504 included in the composition can be genetic data (e.g., DNA, a portion of a genetic dataset, one or more oligonucleotides, other pairs of nucleotides, and/or any other information corresponding to genetics or identity of an organism). For example, the metadata 502 and sample data 504 can include one or more etches or genetic sequences (e.g., to be determined by sequencing), and the metadata 502 can include one or more etches selected to encode one or more contextual parameters associated with the sample data 504 (e.g., a genetic sample). In some implementations, the sample data 504 can include genetic information of a single person or individual, and the metadata 502 can include contextual information (e.g., data of the capture of the sample data or the processing of the sample data, etc.) encoded or combined to describe the sample data 504. For example, two or more predefined metadata sequences can be generated and stored, and the two or more of the predefined metadata sequences can be combined to encode metadata (e.g., metadata 502) describing capture parameters or processing parameters corresponding to the capture (e.g., collection) or processing of the sample data 504. While illustrated as single portions or sequences of DNA on FIG. 5, it should be understood that each of the metadata 502 and/or sample data 504 can include multiple DNA portions (e.g., combinations of sequences, etches, etc.), as shown in detail in FIGS. 5B-5D. Further, sample environment 360 can include multiple containers for storing multiple sets of metadata 502 and sample data 504 (e.g., corresponding to multiple organisms).

The sample environment 360 can include and/or provide a composition including separate metadata 502 and sample data 504. For example, the metadata 502 and sample data 504 can both be contained in the same physical enclosure and/or composition, but metadata 502 and the sample data 504 may not share chemical bonds (e.g., covalent bonds) or be otherwise attached or connected (e.g., being separately maintained or non-covalently pooled). In some implementations, the sample environment 360 can provide the metadata 502 and/or sample data 504 without modifying, deleting from, appending to, altering, perturbing, or otherwise disturbing the sample data 504, thereby providing the sample data 504 to be accurately processed. For example, the metadata 502 and sample data 504 can be provided to a sequencing system via the sample environment 360, and the sequencing system can process and/or sequence the metadata 502 and sample data 504 such that one or more etches of the metadata 502 can be identified and one or more genetic sequences of sample data 504 can be identified.

Still referring to FIG. 5A, the sample environment 360 can be used in a method of providing a sample environment for storing a target genetic sample of an organism with encoded genetic metadata describing the target genetic sample. The method can include adding the target genetic sample of the organism to sample environment 360 (e.g., adding DNA material of the sample). The sample environment 360 can include a container (e.g., test tube, well of a sequencing system, etc.) for storing genetic material. The method of providing the sample environment 360 can further include adding one or more etches of the encoded genetic metadata to the sample environment (e.g., adding two or more genetic etches to encode metadata descriptive of the sample). In some implementations, adding the encoded genetic metadata includes generating a composition by incorporating or including the one or more etches of the encoded genetic metadata (e.g., metadata 502) in the sample environment 360 unbonded with the target genetic sample (e.g., sample data 504). For example, as described above, the genetic metadata 502 and sample data 504 can both physically occupy sample environment 360 without sharing any connection (e.g., bonding) or causing any interaction potentially interfering with subsequent sequencing of either the genetic metadata or target genetic sample (e.g., simultaneously or separately).

Referring now to FIG. 5B, the sample environment 360 can include a composition with multiple sample data sequences 504a-504n and one or more metadata etches 502a. For example, the sample data sequence 504a can be a first genetic sequence of first organism (e.g., Human 1), and the sample data sequence 504n can be second genetic sequence of the first organism (e.g., Human 1) (e.g., the sample environment 360 storing multiple genetic sequences of the same organism). In another example, the sample data sequence 504a can be a first genetic sequence of a first organism (e.g., Human 1), and the sample data sequence 504n can be a first genetic sequence of a second organism (e.g., Canine 1) (e.g., the sample environment 360 storing genetic sequences of multiple organisms of the same or different species). While illustrative examples are provided above, it should be understood that various sample data sequences 504a-504n associated with various DNA of one or multiple organisms can be included and/or pooled (e.g., with metadata) in sample environment 360.

Referring now to FIG. 5C, the sample environment 360 can include a composition with multiple metadata etches 502a-502n. As described below regarding FIGS. 6A-6B, each of the metadata etches 502a-502n can be non-organismal (e.g., non-human, semi-human, or alter-human) genetic sequences (e.g., including nucleotide base pairs and approximately one hundred and fifty base pairs in length, etc.) encoded to represent information related to the sample data 504 or the processing (e.g., sequencing) of the sample data 504. For example, the metadata etches 502a-502n can include sequences that do not interfere with processing of the sample data 504 (e.g., that do not map or align with a standard reference genome of the organism providing the sample) such that sequencing the contents of the sample environment 360 can provide reads (e.g., genetic information, mapped genetic information, etc.) from non-human DNA fragments (e.g., from metadata etches 502a-502n) in addition to reads from human (or human-like) DNA fragments (e.g., from sample data 504). In another example, the metadata etches 502a-502n can include at least four distinct etches encoded uses a binary encoding scheme (e.g., four-bit binary scheme) and pooled separately from the sample data 504.

Referring now to FIG. 5D, the sample environment 360 can include a composition with multiple metadata etches 502a-502n and multiple sample data sequences 504a-504n. In some implementations, the sample data sequences 504a-504n can be multiple sequences associated with the same organism (e.g., with two or more sequences containing genetic information corresponding to the organism). In some implementations, sample data sequences 504a-504n can be multiple sequences associated with different organisms (e.g., with sample data sequence 504a associated with a first organism of a first species and sample data sequence 504b associated with a second organism of the first species, or of a second species). For example, the sample data sequences 504a-504n can include genetic information from diverse sources, including sequences derived from multiple organisms within the same species or even across different species.

Referring generally to FIGS. 6A-6B, flowcharts for a method 600 for generating and incorporating encoded genetic metadata with a target genetic sample of an organism are shown, according to some implementations. One or more systems, sub-systems, or components described herein (e.g., system 100 of FIG. 1, metadata system 330 of FIG. 3, etc.) can perform the steps of method 600 (e.g., via one or more processing circuits). Referring to FIG. 6A in more detail, the method 600 can include determining whether new or updates to etches (e.g., metadata, genetic metadata, nucleotide pairs or sequences, oligonucleotides encoded to represent data, etc.) exist at block 610. As described above, for example, updates to etches can include modifications to encoded genetic metadata, modifications to etches used to generate encoded genetic metadata, etc. If there are no new etches, the method 600 can bypass one or more steps and proceed directly into recording evidence of extracts, dilutions, and etches at block 640. If new etches or updates to etches are determined, the method 600 can continue by resuspending the new etches at step 612, filling out a bit tracking sheet (e.g., Qubit sheet) or other recording document at step 614, and performing a bit or encoding quality check (QC) and/or other QC process at step 616. Although method 600 describes specific quantities and parameters (e.g., volumes, numbers of cycles, etc.), these specific recitations should be construed as illustrative and not limiting. For example, other implementations of the systems and methods described herein can include different quantities and parameters. The provided details and values are intended to facilitate understanding of an embodiment or implementation and should not be construed as limiting. The scope of the invention is defined by the claims rather than the specific examples and values provided herein. Variations and modifications can be made as would be understood by one of ordinary skill in the art, and without departing from the spirit and scope of the invention.

Still referring to FIG. 6A, the method 600 can continue by determining whether the quality check process is passed or not passed (e.g., failed) at block 620. In some implementations, if the QC process is failed, the method 600 can include re-ordering an etch (e.g., from a manufacturer) at block 622 and a post-lab technician verbally notifying or communicating (e.g., via e-mail) with a lab manager at block 624. If a determination is made that the QC process is passed at block 620, the method 600 can include filling out a fragment analyzer sheet or other recording instrument at block 626 and performing fragment analysis (e.g., processing one or more genetic sequences or etches) at block 628.

Further, the method 600 can include determining whether the fragment analysis is passed or not passed (e.g., failed) at block 620. If a determination is made that the fragment analysis is not passed at block 620, the method 600 can include re-ordering the etch at block 632 (e.g., from a manufacturer) and/or a post-lab technician verbally notifying or communicating with a lab manager at block 634, as described above. If the fragment analysis is passed at block 620, the method 600 can continue by saving all QC related data filed to a network location (e.g., to a network share for MT validations) at block 636, further diluting etches based on Qubit and fragment analysis data to reach a working concentration (e.g., a concentration suitable for genetic sequencing and/or other processing) at block 638, and recording evidence of extracts, dilutions, and etches corresponding to a genetic sample in a database or data store at block 640.

Referring now to FIG. 6B in more detail, the method 600 can continue from block 640 as shown in FIG. 6A by selecting one or more etches to encode information at blocks 650-658. While the encoding of certain information is described below, it should be understood that various information related to genetic data (e.g., environmental data, collection data, processing data, etc.) or otherwise (e.g., laboratory identifiers, case numbers, executable codes, etc.) can be encoded using combinations of etches (e.g., synthetic oligonucleotides, etc.). For example, an etch can be selected based on an identifier (e.g., an identifier of the genetics company analyzing or processing the genetic sample) and/or based on genetic tagging, an etch corresponding to a month (e.g., a Month etch) can be selected at block 650, an etch corresponding to a day (e.g., Day etch) can be selected at block 652, an etch corresponding to a sequence (e.g., Sequence etch) can be selected at block 654, an etch corresponding to a flexible data field (e.g., Flex etch) can be selected at block 656, and/or an etch corresponding to a technician (e.g., analyst, operator, user, etc.) (e.g., Initial etch) can be selected at block 658.

Further, the method 600 can include diluting an evidence extract (e.g., an extract containing and/or combining etches above) at block 660 and/or diluting etches at block 662. For example, diluting an evidence extract at block 660 can include diluting based on a dilution factor determined at an input assessment (e.g., performed at a previous step in method 600), and diluting etches at block at block 662 can include diluting based on a type of library preparation. In some implementations, the method 600 further includes determining whether the type of preparation used (e.g., the type of library preparation used in the dilution of etches at block 662) is a default preparation (or default prep) at block 670. If the type of library preparation is a default type, the method 600 can continue by performing a default dilution (e.g., 15.68 microliters of EDTA Tris; one microliter each) at block 672 and photographing a rack containing the DNA extract and all etches to be used for future reference at block 674.

Still referring to FIG. 6B, if it is determined that the type of library preparation is not a default type at block 670, the method 600 can continue by determining whether non-human filtering of the extract is to be performed at block 680. If it is determined that non-human filtering is to be performed at block 680, the method 600 can further include performing a filtering dilution (e.g., 7.83 microliters of EDTA Tris; one microliter each) at block 682 and pooling etches in a separate sample environment (e.g., test tube) at block 684 (e.g., to create a combination of multiple etches that define a tag for a sample). If it is not determined that non-human filtering is to be performed at block 680, the method 600 can further include performing an 8-9 cycle amp dilution (e.g., 31.34 microliters low EDTA Tris; 1 microliter each) at block 686 before pooling the etches in the separate tube at block 684. In some implementations, the method 600 further includes adding the pooled etches to a diluted evidence extract (e.g., target genetic sample, sample environment including a genetic sample, etc.) at block 688. Further, block 690 can include determining whether all evidence extracts are tagged (e.g., by being included with genetic metadata descriptive of the evidence extract and/or genetic information of the evidence extract) and if so, proceeding to an additional step in method 600 (e.g., providing tagged evidence extract for sequencing). If it is determined that all evidence extracts are not tagged at block 690 (e.g., one or more extracts do not have pooled etches), the method 600 can include returning to block 640 and repeating the previous steps as described above.

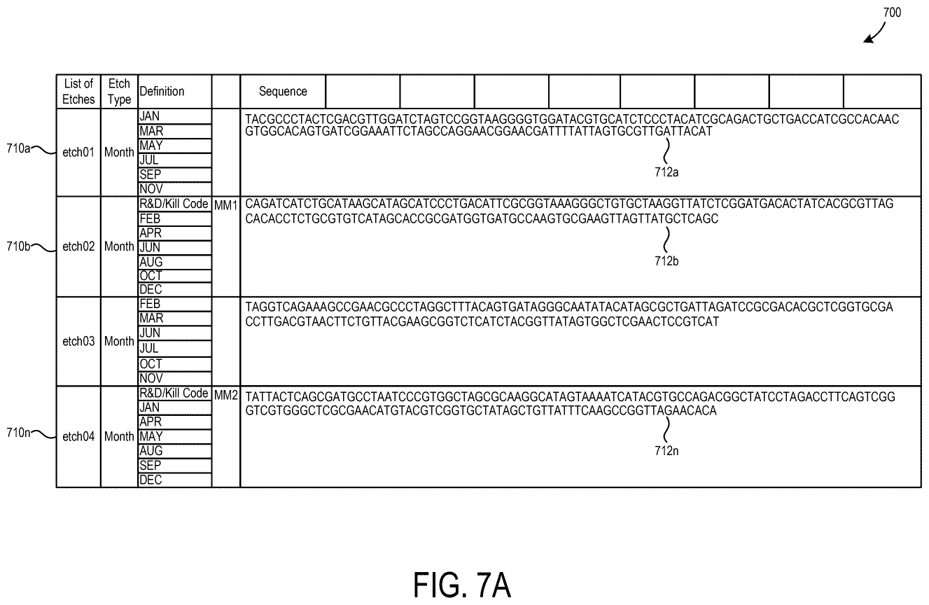

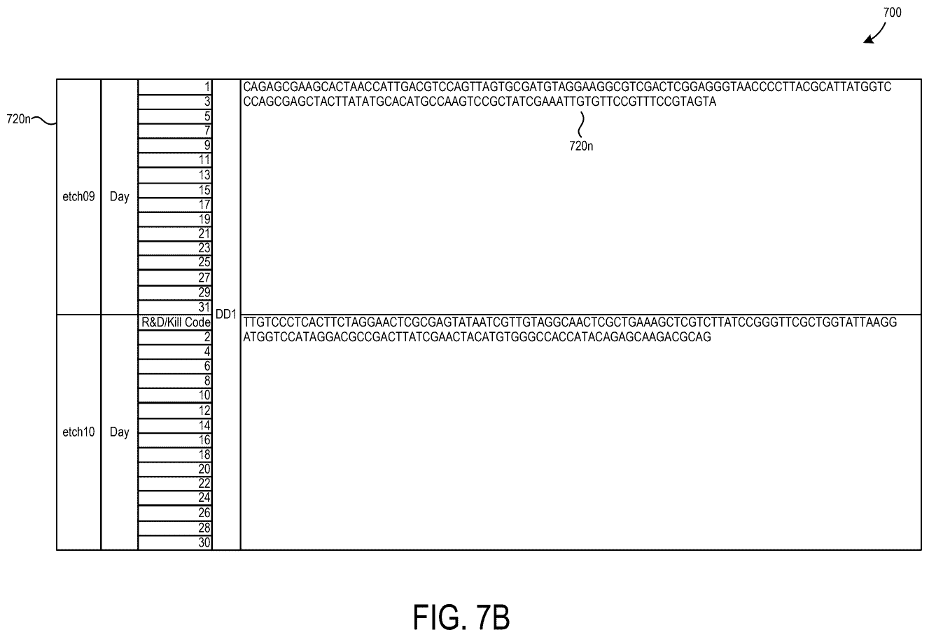

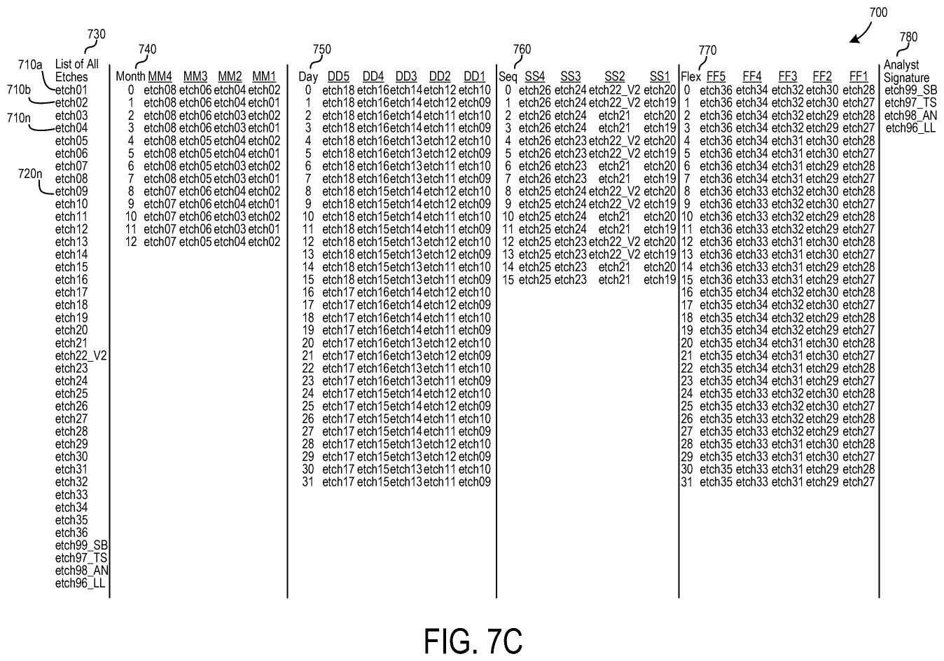

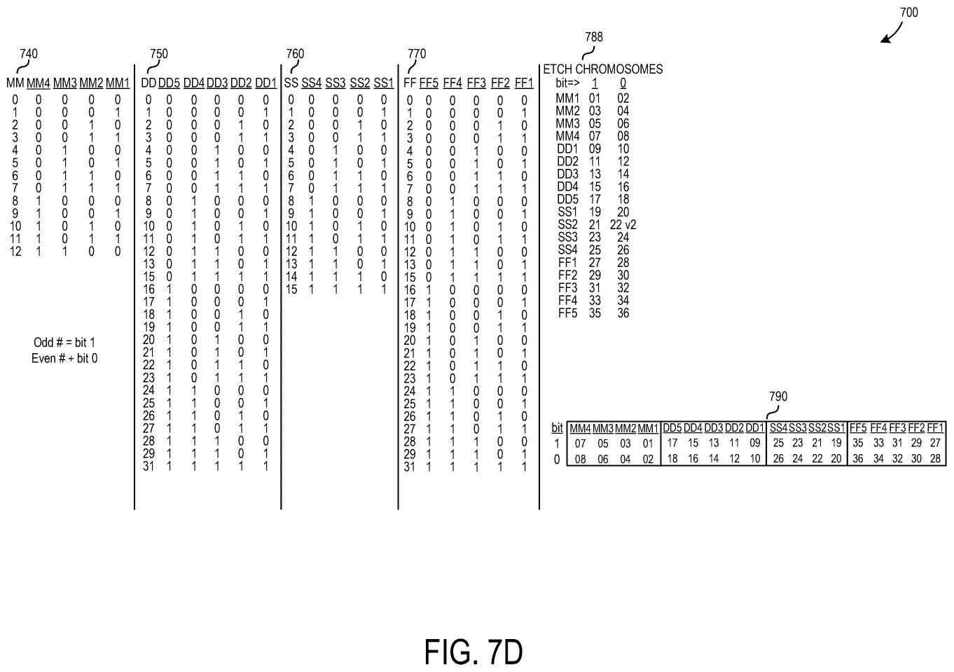

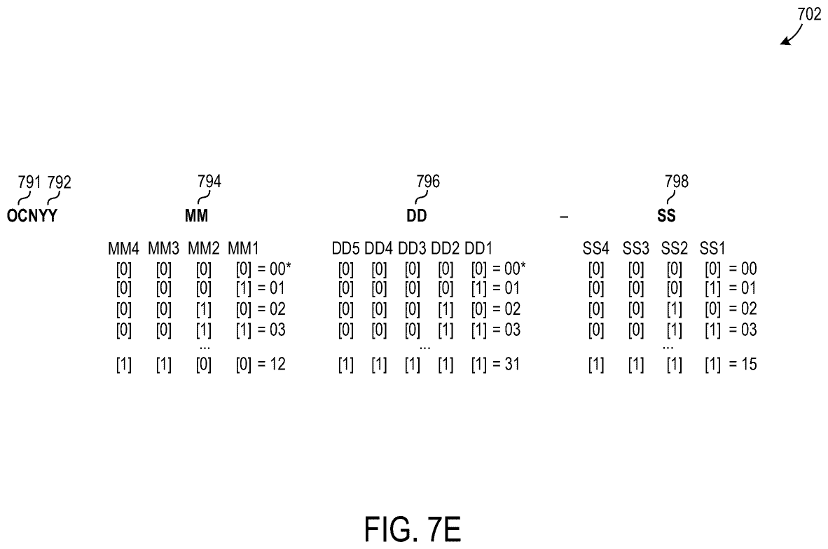

FIGS. 7A-7E are block diagrams depicting examples of genetic metadata and encoding, according to some implementations. Referring to FIG. 7A in greater detail, a library 700 including one or more etches 710a-710n (collectively the etches 710) is shown, according to some implementations. For example, the library 700 (e.g., decoding reference database) can include a list of available (e.g., predefined, generated, etc.) etches for encoding genetic metadata. As shown, each of the etches 710 can include respective genetic sequences 712a-712n (collectively genetic sequences 712) containing one or more pairs of nucleotides (e.g., pairs of adenine (A), thymine (T), guanine (G), and cytosine (C) attached to form chemical bonds) corresponding to each of etches 710a-710n. For example, genetic sequence 712a corresponds to etch 710a and includes a plurality of nucleotides forming a sequence (e.g., beginning with “TACGCC . . . ”). Similarly, genetic sequence 712b corresponds with etch 710b, and so on for a plurality of etches (e.g., etches 710a-710n and genetic sequences 712a-712e).

Still referring to FIG. 7A, each of the etches 710 can correspond to one or more etch types (e.g., for interpretation after encoding), such as a “month” type used for encoding a representation of a month (e.g., a month of sequencing such as January, February, etc., as shown regarding etches 710a-710n), a “day” type used for encoding a date (e.g., collection date, processing date, metadata generation date, etc.), and various other fields or etch types for encoding a variety of data (e.g., analyst initial type, case number, sample identifier, metadata identifier, etc.).

Referring now to FIG. 7B, for example, an etch 720n can correspond to a “day” type and include one or more etches 720n (e.g., synthetic genetic sequences or combinations of nucleic acid or base pairs, as shown beginning with “CAGACG . . . ”). Various other etch types and/or genetic information can be encoded as described above regarding etches 710 of FIG. 7A and/or etch 720 of FIG. 7B, using corresponding sequences (e.g., sequences 712a-712n, etch 720, etc.) or other sequences configured to be interpreted by as information (e.g., as genetic information by a sequencing machine during sequencing).

Referring now to FIG. 7C, the library 700 (e.g., decoding reference database) can further include etch list 730 (e.g., a list of available sequences, etches, tags, etc.) available for use as encoded genetic metadata. In some implementations, the etch list 730 can include a set of available etches (e.g., etches 710a-710n of FIG. 7A, etch 720 of FIG. 7B, etc.), and one or more etches included in the etch list 730 can correspond to and be used to encode information indicative of the type of data represented by an etch type (e.g., day, month, sequencing order, etc.). The etch list 730 can be associated with lists 740-780 containing etches and corresponding categories and/or etch types. For example, month list 740 can include a listing of all etches used to encode information indicative of a “month” (e.g., a month of sequencing such as October, November, etc.).

As shown, month list 740 can include a key or other information indicating an encoding scheme using combinations of one or more etches to represent data related to the “month” type. For example, as shown on FIG. 7C, the month of January (e.g., corresponding to a numeric “1” when describing a calendar year numerically as months 1-12) can be encoded by combining four etches (e.g., etch08, etch06, etch04, and etch01), with each etch corresponding to one bit of information, and with each month encoded as a unique combination of four etches (e.g., bits). For example, list 750 can include a list of etches and/or an encoding scheme for encoding a numeric indicator of a day (e.g., “29” to represent processing of a sample on the 29th day of a month, etc.). As shown on FIG. 7C, for example, the day can be encoded by combining five etches (e.g., etch17, etch15, etch13, etch12, and etch09 to represent the 29th day of a month), with each etch corresponding to one “bit” of information and with each potential date within a given month encoded using a unique combination of five etches. Further, list 760 can include a list of etches and/or encoding schemes to represent a sequencing or processing order (e.g., as a number between 0 and 15), and various sequencing or processing orders can be represented using four bits or etch combinations (e.g., with a value of “2” in the sequencing order being encoded using etch26, etch24, etch21, and etch20), as described above. List 770 can include a list of etches and/or encoding schemes to represent “flex” data (e.g., additional data beyond the data illustrated by the lists 740-760 and list 780, such as laboratory or environmental data). For example, additional pairs of etches can be designated for flexible uses, such as distinguishing among multiple extracts from the same case (e.g., by sequentially assigning numerically ordered labels, starting with the first extract identified by either a unique item number or by its sequence within the same item or sample). Further, list 780 can include a list of etches and/or encoding schemes to represent an analyst signature (e.g., initials of the technician, operator, or analyst generating the metadata or otherwise handling, interacting with, or managing the sample).

Referring now to FIG. 7D, each of the lists 740-770 can include unique combinations of bits (e.g., portions of information represented in Boolean (e.g., true or false), binary (e.g., 1 or 0), etc.). Each of the etches included in the lists 740-770 can correspond to an encoded value (e.g., 1 or 0 using a binary scheme). For example, to represent the 1^{st} month (e.g., January) as defined in list 740, four etches (e.g., sequences preassigned to represent a month type) can be combined by including a first etch indicative of a “0” (e.g., etch08 of FIG. 7C), a second etch indicative of a “0” (e.g., etch06 of FIG. 7C), a third etch indicative of a “0” (e.g., etch04 of FIG. 7C), and a fourth etch indicative of a “1” (e.g., etch01 of FIG. 7C).

Further, each of the lists 750-770 can include similar encoding schemes using combinations of two or more etches (e.g., of the same etch type) to indicate unique information corresponding to the etch type. For example, list 750 can include an encoding scheme for encoding a day and illustrate unique combinations of etches that can be encoded to represent a unique date value (e.g., in groups of five, such as combining etch17, etch15, etch13, etch12, and etch09 to represent the 29th day of a month, as shown on FIG. 7C). Further, the library 700 can include a list 788 of etch types and corresponding bits (e.g., MM1 for the first bit or etch used to encode a month, MM2 for the second etch used to encode the month, MM3 for the third etch, and MM4 for the fourth etch). The library 700 can further include a list 790 of etch types and bits (e.g., MM1-MM4, DD1-DD5, SS1-SS4, FF1-FF5, etc.) including data of which etches of each etch type correspond to a 1 or a 0 using a binary encoding or other encoding scheme. For example, etch07, etch05, etch03, and etch01 can represent a “1” when used to encode a month value, and etch08, etch06, etch04, and etch02 can represent a “0” when used to encode a month value.

Referring now to FIG. 7E, combinations of etches of the same etch type can be combined or appended together to create a unique identifier 702 (e.g., metadata). The unique identifier 702 can include a plurality of etches, and each of the plurality of etches can encode information related to a target genetic sample (e.g., hair sample of a human) to generate the unique identifier 702. In some implementations, the unique identifier 702 includes data such as a laboratory identifier (e.g., shown on FIG. 7C as organizational case number (OCN) 791), a year identifier (e.g., shown as year identifier (YY) 792 on FIG. 7E), a month identifier 794, a day identifier 796, and/or a sequencer identifier 798. For example, etches indicative of values for the laboratory identifier 791, year identifier 792, month identifier 794, day identifier 796, and sequencer identifier 798 can be combined (e.g., by directly appending or by using formatting such as underscore, hyphens, etc. to separate information corresponding to different etch types) to produce the unique identifier 702 (e.g., identification number, key, case number, etc.). In some implementations, the unique identifier uniquely identifies a sample of genetic material (e.g., a DNA sample) by including information descriptive of the sample genetic material (e.g., timestamp of collection, date/time of processing, analyst information, sequencing orders, etc.). A variety of additional etches of various types can be included and combined to encode information as the unique identifier 702 (e.g., by including etches to indicate a court number corresponding to a legal case related to the sample DNA in addition to the laboratory identifier 791, year identifier 792, month identifier 794, day identifier 796, and sequencer identifier 798).

Referring generally to FIGS. 7A-7E, each of the etches (e.g., etches 710 of FIG. 7A, etch01 of FIG. 7B, etc.) can be selected and/or generated to not interfere with subsequent processing (e.g., amplification, sequencing, etc.) of sample genetic material. For example, each of two or more etches of encoded genetic metadata can be pooled with a target genetic sample (e.g., using sample environment 360 of FIGS. 3 and 5), and pooling can maintain the target genetic sample in a processable or genetic-readable format (e.g., providing the sample genetic material to be sequenced to determine a genetic profile of the organism without the metadata interfering with accurate processing/sequencing of the sample). In some implementations, the target genetic sample of the organism includes DNA of a human organism, and the one or more etches of the encoded genetic metadata include at least one non-human sequence (e.g., one or more oligonucleotides or other combinations of base pairs that do not map to the human reference genome when sequenced). For example, the one or more etches of the encoded genetic metadata can diverge from (e.g., not align with, not map to, etc.) a reference genome of the organism providing the sample.

In some implementations, the organism providing the sample can be a human, and in others, the organisms providing the sample can be another animal (e.g., dog, cat, etc.) or plant (e.g., flower, fungus, etc.). In implementations involving a human DNA sample, for example, the etches can be used to encode metadata (and/or the combined etches forming the metadata) can be selected such that the etches/sequences do not map to the human reference genome when sequenced. In implementations involving DNA samples from non-human organisms (e.g., from canines, felines, or other animals; from plants; from other genetic material used as etches; etc.), the etches can similarly diverge from a reference genome of the organism (e.g., diverging from a mapped genome of a canine for implementations including canine sample DNA, etc.) and/or otherwise not interfere with accurate processing of the sample.

Still referring to FIGS. 7A-7E, various encoding schemes for encoding genetic data (e.g., using etches) can be employed. For example, a binary system of identifiers can efficiently identify one or more metadata values using various combinations of etches as “bits,” as described above. However, to avoid etch dropout or tag dropout (e.g., failure to recover reads for any etch present in an extract for stochastic or other reasons, etc.), the encoding scheme presented herein can include representing binary “0” and “1” values by alternate etches, rather than including binary values being represented the absence/presence of a single etch. For example, rather than assigning the absence of a given etch to represent a “0” value, one or more different etches of a given etch type can be assigned to represent the “0” value. Accordingly, when etches (e.g., etch01 or etch 710a of FIG. 7A, or combinations of etches as shown on FIGS. 7A-7E) are sequenced using the systems and methods described above, genetic analysis accuracy can be improved by avoiding misidentification associated with typical binary schemes that use an absence of a data portion to represent a bit (e.g., a “0”). Further, sequencing accuracy can be improved by including pre-defined (e.g., pre-mixed, prepared, etc.) etches for each month, day, and sequential number, and by identifying these etches using human-readable decimal equivalents (e.g., by using a “2” to represent the second month of a year, February). The disclosed systems and methods can also include verifying data encoded as metadata (e.g., using the modified binary scheme described above) by storing corresponding data captured through scanning, photography, and/or witnessing, and by comparing the stored data corresponding to the metadata (e.g., simultaneously with pooling the metadata and sample, at a later time after pooling and/or sequencing, etc.) to the metadata to avoid errors in analysis.

In some implementations, in addition to the encoding schemes illustrated above, various alternative methods can be employed to encode genetic metadata for various purposes (e.g., for enhancing robustness of data, for improving efficiency of data encoding). For example, differential encoding or a differential encoding technique can be used, and each bit can be represented by a relative quantity of two etches. For example, if etch08 and etch06 from FIG. 7C are used in a differential encoding scheme, a higher quantity of etch08 can signify a ‘0,’ while a higher quantity of etch06 can signify a ‘1.’ Differential encoding can provide increased noise tolerance by mitigating the impact of unintended DNA fragments aligning with the etch sequences and introducing redundancy. Further, encoding schemes such as 8b10b can be utilized to define a number of etches for binary encoding. For example, 8b10b encoding can convert 8 bits of data into 10 bits such that no more than five etches are present in any given sample. Additionally, Manchester encoding can be applied, where each bit of information is represented by a transition between etches such that the encoded data is not misinterpreted (e.g., due to signal degradation). For example, a transition from etch08 to etch06 can represent a ‘1,’ while a transition from etch06 to etch08 can represent a ‘0.’ While examples are described above, it should be understood that various additional and/or alternative encoding techniques, schemes, or strategies can be implemented in various embodiments of the present disclosure. For example, encoding schemes can include, but are not limited to: Manchester encoding, Differential encoding, Non-Return-to-Zero Inverted (NRZI) encoding, Pulse-code modulation (PCM), Binary Phase Shift Keying (BPSK), Miller encoding, 8b/10b encoding, 6b/8b encoding, binary-to-text encoding, one-hot encoding, label encoding, character encoding, HTML encoding, URL encoding, Unicode encoding, Base64 encoding, Hex encoding, ASCII encoding, hashing encoding, and more.

In some implementations, error detection mechanisms (e.g., error detection codes) can be incorporated to improve the integrity of the encoded genetic metadata. For example, a parity bit can be utilized. In this example, a parity bit can be a bit of information deriving value from the sum of the other bits in the encoded data. The parity bit can be used to detect errors such as missing or altered etches. For example, if the sum of the bits in the encoded data is even, the parity bit can be set to ‘,’ and if odd, the parity bit can be set to ‘1.’ Upon decoding, the parity bit can be recalculated and compared to the original parity bit to verify data integrity. A discrepancy between the recalculated parity bit and the original parity bit can indicate an error in the encoded data, such as the absence or alteration of one or more etches.

In addition to parity bits, any of a variety of other error detection techniques or frameworks (e.g., checksums, hash functions, etc.) can be employed. For example, a checksum can be computed as a numerical value representing the sum of the bits in the encoded data. The checksum value can be appended to the encoded data and verified upon decoding to verify that the data has not been altered. Similarly, a hash function can be applied to the encoded data to produce a fixed-size hash value that uniquely represents the original data. Any modification to the encoded data can result in a different hash value upon recalculation, thereby indicating the presence of an error. These error detection mechanisms, including parity bits, checksums, and hash functions, can enhance the reliability and accuracy of the genetic metadata encoded using the etches.

Further, the use of error-correcting codes (ECC) can be implemented to allow the information to be successfully decoded even if some of the encoded information is missing. For example, techniques such as forward error correction and erasure codes can be employed to recover information from genetic metadata (e.g., etches) despite one or more of the etches in the sequenced data being absent. In some implementations, ECC methods can include adding extra etches or bits to the encoded information, and the extra etches or bits can significantly increase the likelihood of successfully recovering the encoded data. For example, if certain etches are filtered out or are insufficiently represented in the sequenced data, the missing information can still be reconstructed using the error-correcting codes (e.g., using digital data storage and transmission techniques modified to handle genetic data, such as modified versions of techniques employed in ECC RAM and CD/DVD technology).

In some implementations, invalid or incorrect decoded etches can be addressed by specific processes to recover missing data or otherwise improve data integrity. For example, if a decoded sample indicates an impossible or suspect date, such as the 32nd of January, the sample can be subjected to additional verification procedures. One approach can involve comparing the invalid sample against all possible samples included in the same sequencing run to identify a closest match with the fewest bit-flips, thereby determining the correct data. Another method can involve cross-referencing other encoded etches within the same batch to identify missing samples and resolve discrepancies. In some arrangements, the processes described above can further include re-sequencing or re-reading the genetic metadata, analyzing the metadata, and consulting laboratory records to mitigate errors and provide accurate results from genetic analysis.

The various encoding, error-detection, error-correction, and other error-checking techniques described herein, including differential encoding, 8b10b encoding, Manchester encoding, parity bits, checksums, hash functions, and other error-detection or error-correcting codes, illustrate a variety of methods for reliably encoding and retrieving bits in DNA samples. It is important to note that the encoding and error-checking techniques described herein are not exhaustive. Other methods and variations can be utilized to achieve similar results. For example, Hamming codes, Reed-Solomon codes, Low-Density Parity-Check (LDPC) codes, turbo codes, and Cyclic Redundancy Check (CRC) codes can be used as error-correcting codes to identify, determine, and/or output errors or discrepancies in sequenced encoded genetic metadata (e.g., after sequencing). In another example, various encoding schemes such as Differential encoding, Non-Return-to-Zero Inverted (NRZI) encoding, Pulse-code modulation (PCM), Binary Phase Shift Keying (BPSK), Miller encoding, and 8b/10b encoding can be implemented. The various encoding and error-checking approaches described herein can be applied to a range of genetic metadata encoding or sequencing scenarios, as illustrated in FIGS. 7A-7E, or otherwise.

In some implementations, etches (e.g., synthetic nucleic acid sequences) can include cloneable, blunt-ended, double-stranded synthetic DNA molecules and can be 150 base pairs in length. To avoid interference with subsequence processing, strings with equal representation of A, C, G, & T (37, 38, 38, & 37, respectively) can be developed and/or scrambled, and any strings with homopolymers greater than four nucleotides long or “GC” portions/runs of greater than five nucleotides can be discarded (e.g., not included as an etch or within an etch library, etc.).

Further, to provide sufficient sequence distance from DNA of an organism corresponding to the sample (e.g., from a human in examples involving human DNA samples) and to provide distance from other natural components of casework (e.g., to prevent cross-mapping), a plurality of candidate etch sequences (e.g., approximately 4000 or more) can be searched against the human reference genome (e.g., BLAST vs GRCh38.p12) using various parameters (e.g., with a word size of 11, expect threshold of 10, all filters disengaged, etc.) to determine candidate etches that do not align with the reference genome. Sequences with any match to a human can be discarded, and a remainder of etches can be searched (e.g., against the nr/nt database of non-redundant sequences (roughly 58,021,211 sequences), etc.) with the same parameter settings or different settings (e.g., lower effective sensitivity due to differences in database size). Candidate etch sequences with any match to nr/nt can be discarded, leaving a final set of candidate etch sequences of the initial set with no similarity to known sequences. In some implementations, one or more of these sequences can be modified to encode (using the standard genetic code) various information (e.g., to encode analyst initials in the first 6-9 nucleotides (top strand) without altering sequence composition). The candidate etches can be further verified for uniqueness against a human and/or against nr/nt, as described above, and candidate etches meeting the stated criteria can be assigned an etch type (e.g., month, day, etc.), included in the library 700, and/or be otherwise selected to be combined with other etches to be encoded as genetic metadata.

Similar processes can be performed for various organisms (e.g., canines or other animals, plants, etc.) such that selected etches do not interfere with processing of the sample DNA during sequencing (e.g., providing the sample DNA and the genetic metadata to be accurately sequenced). Further, because off-target products (e.g., etches/sequences with incorrect base pairs or otherwise containing deficiencies) can include large truncations that could cause issues for sequencing if present in large quantities, etches can be synthesized such that 90% or more of etch clones (e.g., genetic copies received from a manufacturer, DNA fragments produced during PCR, etc.) accurately include an original pre-defined sequence. In some implementations, the accuracy of etches can be improved using base size selection and/or using a fragment analyzer.

Still referring to FIGS. 7A-7E, etch design validation can include a preliminary screening to evaluate the likely performance and robustness of the candidate etches. For example, a portion of etch data (e.g., 10,000 150 bp read pairs) can be drawn from a control sequencing (e.g., fastq) dataset, identifiers can be modified, sequences can be stripped and replaced with a predefined number of copies (e.g., 10,000 copies) of each candidate sequence, and the sequences can be trimmed to match the variable original adapter-trimmed lengths of each read and/or quality string. Further, sequencing errors can be simulated (e.g., at a predefined or dynamic rate) to yield possible substitutions at various positions for a predefined number of times (e.g., a least 10 times), with or without regard to the associated quality strings (e.g., without regard to the quality strings to simulate a non-ideal or worse-case scenario). In some implementations, outputted read pairs can be concatenated to a hybrid raw sequence dataset (e.g., a dataset consisting of a series of 10 million read samples from different forensic-type inputs). This dataset can be mapped (e.g., using standard pipeline parameters) to a modified reference including candidate sequences appended as separate chromosomes. Out of the predefined (e.g. 10,000) read pairs for each sequence, some number of reads derived from etches can failed to map to corresponding reference chromosomes, and etches that did not map to the correct chromosome (e.g., 35 bp long truncated reads) can be included in a predefined etch library for encoding metadata as combinations of etches. The verification process can further include determining whether no primary, secondary, or supplemental alignments of etches align or read to any other tag sequence, nor to anywhere in the human genome, as well as determining other genetic information related to the candidate etches (e.g., determining that only non-etch reads that mapped to etches were short 19-21 base alignments that are effectively removed by standard filters, etc.).

Still referring generally to FIGS. 7A-7E, one or more etches (e.g., etches 710a-710n of FIG. 7A) can be encrypted, include encrypted data, or include data or information associated with encryption (e.g., as encoded metadata of target genetic sample). For example, textual or numerical data (e.g., processing parameters, capture event parameters, etc.) can be encrypted directly (e.g., using a key-based algorithm), and combinations of one or more etches 710a-710n can be assigned to represent the encrypted textual or numerical data (e.g., by being added to an encryption database or etch library used for interpreting or decoding encoded metadata). The encrypted textual or numerical data can further be encoded into genetic metadata (e.g., as combinations of one or more etches 710a-710n), the encrypted data can be determined by sequencing (e.g., using a synthetic genome as a reference genome), and the sequenced encrypted data can be decoded (e.g., using a stored dataset or library to determine the string of encrypted textual or numerical data corresponding to sequenced etches, and using an encryption key or database to decode the encrypted textual or numerical data). While an illustrative encryption scheme is outlined above, it should be understood that various additional and/or alternative encryption methods can be implemented for encoding metadata in a secure format (e.g., employing genetic variations of symmetric encryption techniques such as secure hash algorithm (SHA) or advanced encryption standard (AES) methods, asymmetric methods such as RSA or homomorphic encryption, and/or other encryption techniques configured to manage the complexity and sensitivity associated with genetic data). For example, AES encryption can be performed to encrypt textual or numerical data associated with genetic metadata. The encrypted data can then be encoded into genetic etches, and upon sequencing, the encrypted information can be decoded using the appropriate encryption key, providing access to the original data while maintaining confidentiality. In implementations that involve both physical data (e.g., physical genetic etches/sequences) and associated virtual data (e.g., stored electronic data corresponding to the etches/sequences), encryption can be applied to the physical data, the virtual data, both, or neither, depending on specific security requirements and/or other factors.

FIGS. 8A-8B are example illustrations depicting a system 800 for receiving and storing genetic metadata, according to some implementations. Referring to FIG. 8A in more detail, an extract dilution recording system 800a can include a table or other data storage structure to store information related to extracting a target genetic sample and/or genetic metadata descriptive of the target genetic sample. For example, as shown on FIG. 8A, the extract dilution recording system 800a can include data related to extract identifiers (e.g., “Extract ID”), one or more quality or other checks performed (e.g., “Extract BC Check”), chemical or concentration information related to the sample or metadata and/or to diluting the sample or metadata (e.g., “Extract Concentration,” “Dilution Factor,” “Diluted Concentration,” etc.), volume and/or quantity information (e.g., “Total DNA Input,” “Volume of DNA Needed,” “Volume of Tris Needed,” etc.), and/or library information (e.g., “Library BC Check,” Library ID”). Data included in the extract dilution recording system 800a can be used for verification or otherwise for various purposes (e.g., for generating encoded genetic metadata and/or capturing data related to the generation of metadata, etc.).

Referring to FIG. 8B in more detail, a molecular etch recording system 800b is shown, according to some implementations. The molecular etch recording system 800b can include a table or other data storage structure to store information related to extracting a target genetic sample and/or genetic metadata descriptive of the target genetic sample, as described above regarding FIG. 8A. For example, the molecular etch recording system 800b can include data related to molecular metadata (e.g., data of etches, encoded genetic metadata, etc.), such as library information (e.g., “Library ID”), extract information (e.g., “Extract BC Check”), etch information corresponding to a given etch type (e.g., “Month,” “Day,” “Sequential,” “Initial,” “Flex,” etc.), concentration checks or other evaluations related to the etches and/or etch types (e.g., “Month BC check,” “Day BC check,” “Sequential BC Check,” “Flex BC Check,” etc.), and/or other information (e.g., a “Witness” or other party verifying the stored information, etc.). Data included in the extract dilution recording system 800a can be used for verification or otherwise for various purposes (e.g., for referencing to determine information encoded as metadata included with a target genetic sample, for updating genetic metadata, etc.).

In some implementations, one or more of the methods, processes, tasks, or operations described herein (e.g., one or more steps of method 200 of FIG. 2, method 400 of FIG. 4, method 600 of FIGS. 6A-6B, etc.) can be performed using a kit. In some implementations, one or more of the systems, sub-systems, or components described herein (e.g., system 100 of FIG. 1, system 300 of FIG. 3, etc.) can perform one or more of the methods, processes, tasks, or operations described herein. A kit can refer to or include a package or collection of physical components or material and/or accompanying software or digital resources that are configured for use in sample preparation, metadata encoding, and/or metadata decoding workflows for genetic samples. For example, a kit for verifying genetic samples using contextual metadata can include a predefined set of etches configured to encode one or more contextual parameters associated with at least one genetic sample and a decoding reference database including mappings between the predefined set of etches and corresponding metadata values of the one or more contextual parameters.

For example, the kit can include etches supplied in individual tubes, pre-aliquoted plates, lyophilized formats, or master mixes suitable for addition to a reaction vessel, sequencing well, or other sample environment including genetic material. The kit can include a decoding reference database supplied as a local file (e.g., JSON, CSV, or proprietary format), a web-accessible database, or an application program interface (API) that can be queried by a client device or system. In some implementations, the kit can additionally include protocols or written instructions specifying how to pool the etches with the genetic sample, perform downstream sequencing, and/or interpret results using the decoding reference database. The kit can further include control samples, buffers, or other reagents for library preparation or quality control. In some implementations, the kit can be configured for use in laboratory settings such as forensic labs, clinical genomics facilities, or research institutions, and can be used in conjunction with automated or manual workflows. In some examples, the kit can integrate with a laboratory information management system (LIMS) or provide data (e.g., metadata exports) for downstream analysis, audit tracking, or regulatory documentation. The components of the kit can support single-sample or multiplexed workflows. In some examples, the predefined etch set included in the kit can be selected based on various factors associated with an expected use case (e.g., for compatibility with a sequencing systems or platform, based on metadata preferred to be tracked for a given use case, etc.).

Referring now to FIG. 9, a depiction of a computer system 900 is shown. The computer system 900 that can be used, for example, to implement system 100, encoding system 110, readout system 130, decoding system 140, system 300, user device(s) 310, metadata system 330, data sources 350, sequencing system 370, and/or various other example systems described in the present disclosure. The computing system 900 includes a bus 905 or other communication component for communicating information and a processor 910 coupled to the bus 905 for processing information. The computing system 900 also includes main memory 915, such as a random-access memory (RAM) or other dynamic storage device, coupled to the bus 905 for storing information, and instructions to be executed by the processor 910. Main memory 915 can also be used for storing position information, temporary variables, or other intermediate information during execution of instructions by the processor 910. The computing system 900 can further include a read only memory (ROM) 920 or other static storage device coupled to the bus 905 for storing static information and instructions for the processor 910. A storage device 925, such as a solid-state device, magnetic disk, or optical disk, is coupled to the bus 905 for persistently storing information and instructions.

The computing system 900 can be coupled via the bus 905 to a display 935, such as a liquid crystal display, or active matrix display, for displaying information to a user. An input device 930, such as a keyboard including alphanumeric and other keys, can be coupled to the bus 905 for communicating information, and command selections to the processor 910. In another arrangement, the input device 930 has a touch screen display 935. The input device 930 can include any type of biometric sensor, a cursor control, such as a mouse, a trackball, or cursor direction keys, for communicating direction information and command selections to the processor 910 and for controlling cursor movement on the display 935.

In some implementations, the computing system 900 can include a communications adapter 940, such as a networking adapter. Communications adapter 940 can be coupled to bus 905 and can be configured to communicate with a computing or communications network 320 and/or other computing systems. In various illustrative implementations, any type of networking configuration can be achieved using communications adapter 940, such as wired (e.g., via Ethernet), wireless (e.g., via Wi-Fi, Bluetooth), satellite (e.g., via GPS) pre-configured, ad-hoc, LAN, WAN.

According to various implementations, the processes that effectuate illustrative implementations that are described herein (e.g., method 200 of FIG. 2, method 400 of FIG. 4, method 600 of FIG. 6, etc.) can be achieved by the computing system 900 in response to the processor 910 executing an arrangement of instructions contained in main memory 915. Such instructions can be read into main memory 915 from another computer-readable medium, such as the storage device 925. Execution of the arrangement of instructions contained in main memory 915 causes the computing system 900 to perform the illustrative processes described herein. One or more processors in a multi-processing arrangement can also be employed to execute the instructions contained in main memory 915. In alternative implementations, hard-wired circuitry can be used in place of or in combination with software instructions to implement illustrative implementations. Thus, implementations are not limited to any specific combination of hardware circuitry and software.

That is, although an example processing system has been described in FIG. 9, arrangements or implementations of the subject matter and the functional operations described in this specification can be carried out using other types of digital electronic circuitry, or in computer software (e.g., application, blockchain, distributed ledger technology) embodied on a tangible medium, firmware, or hardware, including the structures disclosed in this specification and their structural equivalents, or in combinations of one or more of them. Arrangements of the subject matter described in this specification can be implemented as one or more computer programs, e.g., one or more subsystems of computer program instructions, encoded on one or more computer storage medium for execution by, or to control the operation of, data processing apparatus. Alternatively, or in addition, the program instructions can be encoded on an artificially generated propagated signal, e.g., a machine generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate components or media (e.g., multiple CDs, disks, or other storage devices). Accordingly, the computer storage medium is both tangible and non-transitory.

Although shown in the arrangements of FIG. 9 as singular, stand-alone devices, one of ordinary skill in the art will appreciate that, in some arrangements, the computing system 900 can include virtualized systems and/or system resources. For example, in some arrangements, the computing system 900 can be a virtual switch, virtual router, virtual host, virtual server. In various arrangements, computing system 900 can share physical storage, hardware, and other resources with other virtual machines. In some arrangements, virtual resources of the network 320 (e.g., network 320 of FIG. 3) can include cloud computing resources such that a virtual resource can rely on distributed processing across more than one physical processor, distributed memory, etc.

The systems and methods described herein can include one or more additional embodiments or implementations without departing from the scope herein. Such embodiments, can include, for example, a method for generating encoded genetic metadata, and including the encoded genetic metadata with a target genetic sample of an organism in a sample environment, the method including receiving the target genetic sample of the organism corresponding with capture event parameters, identifying one or more processing event parameters for processing the target genetic sample, generating or obtaining or identifying encoded genetic metadata of the target genetic sample, wherein the encoded genetic metadata includes one or more etches, and wherein generating or obtaining or identifying the encoded genetic metadata includes encoding data of at least one of the capture event parameters or at least one of the processing event parameters as the one or more etches of the encoded genetic metadata, pooling the one or more etches of the encoded genetic metadata with the target genetic sample, wherein pooling includes including the one or more etches of the encoded genetic metadata with the target genetic sample in the sample environment, providing the target genetic sample and encoded genetic metadata, and wherein pooling maintains the target genetic sample in a processable or genetic-readable format.

In some embodiments, the target genetic sample of the organism includes DNA of a human organism, and wherein the one or more etches of the encoded genetic metadata include at least one non-human or non-interfering sequence.

In some embodiments, the one or more etches of the encoded genetic metadata diverge from a reference genome of the organism.

In some embodiments, the one or more capture event parameters includes one or more from a group consisting of a crime scene location, an officer identity, a suspect identity, a victim identity, a time, a timestamp of capture, a crime scene parameter, a unique reference number, a case number, a chain of custody, a crime type, and witness statements.

In some embodiments, the one or processing event parameters includes one or more from a group consisting of processing instructions, sequencing options, testing protocols, preservatives, unique reference numbers, case numbers, operator identity, training records, processing locations, times, timestamps of processing, quality assurance checks, compliance standards, calibration data, equipment use data, consumables and reagents tracking data, processing results, error logs, and data integrity measures.

In some embodiments, the sample environment including the target genetic sample and the encoded genetic metadata is a physical environment and a virtual environment.

In some embodiments, the physical environment is a sample container, and wherein the virtual environment is an electronic storage device.

In some embodiments, the organism is a human, and in some embodiments, the organism is a non-human organism.

In some embodiments, providing further includes providing the encoded genetic metadata and target genetic sample to an evidentiary system or a healthcare system.

In some embodiments, the method further includes receiving, by one or more processing circuits, data corresponding to the generation of the encoded genetic metadata, and storing, by the one or more processing circuits, the data corresponding the generation of the encoded genetic metadata in an electronic storage environment.

In some embodiments, the method further includes determining, by the one or more processing circuits and based on the stored data corresponding to the generation of the encoded genetic metadata, an update to the genetic metadata based on comparing data including the encoded genetic metadata to new data, generating updated encoded genetic metadata, wherein generating includes combining two or more etches stored in a predefined library to represent the new data, and updating, by the one or more processing circuits, the stored data to represent the new data.

In some embodiments, generating the encoded genetic metadata further includes synthesizing or obtaining or identifying two or more etches diverging from a human reference genome, and combining at least two of the two or more etches to represent at least one of the capture event parameters or processing event parameters as at least one non-human or non-interfering genetic sequence.

In some embodiments, generating the encoded genetic metadata further includes implementing an encoding scheme, and wherein the encoding scheme includes one or more from the group consisting of Manchester encoding, Differential encoding, Non-Return-to-Zero Inverted (NRZI) encoding, Pulse-code modulation (PCM), Binary Phase Shift Keying (BPSK), Miller encoding, 8b/10b encoding, 6b/8b encoding, binary-to-text encoding, one-hot encoding, label encoding, character encoding, HTML encoding, URL encoding, Unicode encoding, Base64 encoding, Hex encoding, ASCII encoding, and hashing encoding.

Some embodiments can include, for example, a system for generating encoded genetic metadata, and including the encoded genetic metadata with a target genetic sample of an organism in a sample environment, the system including a receiving system configured to receive the target genetic sample of the organism corresponding with capture event parameters, an identification system configured to identify one or more processing event parameters for processing the target genetic sample, a generation system or obtaining system or identification system configured to generate or obtain or identify encoded genetic metadata of the target genetic sample, wherein the encoded genetic metadata includes one or more etches, and wherein generating or obtaining or identifying the encoded genetic metadata includes encoding data of at least one of the capture event parameters or at least one of the processing event parameters as the one or more etches of the encoded genetic metadata, a pooling system configured to pool the one or more etches of the encoded genetic metadata with the target genetic sample, wherein pooling includes including the one or more etches of the encoded genetic metadata with the target genetic sample in the sample environment, a providing system configured to provide the target genetic sample and encoded genetic metadata, and wherein pooling maintains the target genetic sample in a processable or genetic-readable format.

In some embodiments, the system can further include one or more processing circuits, wherein one or more of the receiving system, the identification system, the generation system or obtaining system or identification system, and the providing system utilize instructions executable by the one or more processing circuits.

In some embodiments, the one or more processing circuits are further configured to analyze the target genetic sample and encoded genetic metadata using the one or more processing event parameters, wherein the one or more processing circuits are configured to receive the target genetic sample and encoded genetic metadata via the sample environment, and sequence the target genetic sample and encoded genetic metadata.

In some embodiments, the one or more processing circuits are further configured to receive sequenced encoded genetic metadata from sequencing the target genetic sample and encoded genetic metadata, and in response to receiving the sequenced encoded genetic metadata, determine at least one error corresponding to the encoded genetic metadata by executing an error-detection code or an error-correction code, wherein the error-correction code analyzes the sequenced encoded genetic metadata to identify and output the at least one error.

In some embodiments, the error-detection code or the error-correction code includes one or more from a group consisting of: Hamming codes, Reed-Solomon codes, Low-Density Parity-Check (LDPC) codes, turbo codes, Cyclic Redundancy Check (CRC) codes, Bose-Chaudhuri-Hocquenghem (BCH) codes, RS232, Ethernet, TCP, UDP, Golay codes, Goppa codes, Viterbi decoders, multidimensional parity codes, checksum codes, hash codes, message authentication codes, alternant codes, AN codes, Berger codes, forward error correction codes, generalized minimum-distance codes, rank error-correction codes, and remote error indication codes.

In some embodiments, the one or more processing circuits are further configured to sequence the target genetic sample and encoded genetic metadata using a synthetic reference genome, wherein the synthetic reference genome includes a digital genetic sequence, and wherein the digital genetic sequence aligns or maps with the one or more etches of the encoded genetic metadata.

In some embodiments, the techniques described herein relate to a system, wherein the one or more processing circuits are further configured to compare the one or more etches of the sequenced encoded genetic metadata to a genetic reference dataset to determine at least one of the capture event parameters or processing event parameters encoded in the genetic material metadata, and determine the determined at least one capture event parameter or processing event parameter aligns with at least one expected capture event parameter or expected processing event parameter.

In some embodiments, the generation system is further configured to generate one or more reagents for amplifying the encoded genetic metadata, wherein generating includes synthesizing or obtaining or identifying one or more genetic primers or probes to selectively bind to at least one of the one or more etches of the encoded genetic metadata, and applying the one or more reagents to the encoded genetic metadata via the sample environment.

In some embodiments, the target genetic sample of the organism includes DNA of a human organism, and wherein the one or more etches of the encoded genetic metadata include at least one non-human or non-interfering sequence.

In some embodiments, the one or more etches of the encoded genetic metadata diverge from a reference genome of the organism.

In some embodiments, the one or more capture event parameters includes one or more from a group consisting of a crime scene location, an officer identity, a suspect identity, a victim identity, a time, a timestamp of capture, a crime scene parameter, a unique reference number, a case number, a chain of custody, a crime type, and witness statements.

In some embodiments, the one or processing event parameters includes one or more from a group consisting of processing instructions, sequencing options, testing protocols, preservatives, unique reference numbers, case numbers, operator identity, training records, processing locations, times, timestamps of processing, quality assurance checks, compliance standards, calibration data, equipment use data, consumables and reagents tracking data, processing results, error logs, and data integrity measures.

In some embodiments, the sample environment including the target genetic sample and the encoded genetic metadata is a physical environment and a virtual environment.

In some embodiments, the physical environment is a sample container, and in some embodiments, the virtual environment is an electronic storage device.

In some embodiments, the organism is a human, and in some embodiments, the organism is a non-human organism.

In some embodiments, the providing system is further configured to provide the encoded genetic metadata and target genetic sample to an evidentiary system or a healthcare system.

In some embodiments the receiving system is further configured to receive data corresponding to the generation of the encoded genetic metadata, and store the data corresponding to the generation of the encoded genetic metadata in an electronic storage environment.

Some embodiments can include, for example, one or more non-transitory computer-readable storage media (CRM) having instructions stored thereon that, when executed by a one or more processing circuits, cause the one or more processing circuits to receive a target genetic sample of an organism corresponding with capture event parameters, identify one or more processing event parameters for processing the target genetic sample, generate or obtain or identify encoded genetic metadata of the target genetic sample, wherein the encoded genetic metadata includes one or more etches, and wherein generating or obtaining or identifying the encoded genetic metadata includes encoding data of at least one of the capture event parameters or at least one of the processing event parameters as the one or more etches of the encoded genetic metadata, provide the target genetic sample and encoded genetic metadata; and wherein the one or more etches of the encoded genetic metadata are pooled with the target genetic sample in a sample environment, and wherein the sample environment maintains the target genetic sample in a processable or genetic-readable format.

In some embodiments, the one or more non-transitory CRM have additional instructions stored thereon that, when executed by the one or more processing circuits, cause the one or more processing circuits to sequence pooled contents of the sample environment including the one or more etches of the encoded genetic metadata and the target genetic sample.

Some embodiments can include, for example, a method of providing a sample environment for storing a target genetic sample of an organism with encoded genetic metadata associated with the target genetic sample, the method including adding the target genetic sample of the organism to a sample environment, wherein the sample environment includes a container for storing genetic material, adding one or more etches of the encoded genetic metadata to the sample environment, wherein adding includes including the one or more etches of the encoded genetic metadata in the sample environment unbonded with the target genetic sample, and providing the sample environment including the one or more etches of the encoded genetic metadata and the target genetic sample to a sequencing system.

In some embodiments, adding the one or more etches of the encoded genetic metadata to the sample environment further includes adding the one or more etches of the encoded genetic metadata to a metadata sample environment, and adding one or more etches of the encoded genetic metadata from the metadata sample environment to the sample environment, wherein adding includes adding the one or more etches of the encoded genetic metadata separately from the one or more genetic sequences of the target genetic sample.

While this specification contains many specific implementation details and/or arrangement details, these should not be construed as limitations on the scope of any inventions or of what can be claimed, but rather as descriptions of features specific to particular implementations and/or arrangements of the systems and methods described herein. Certain features that are described in this specification in the context of separate implementations and/or arrangements can also be implemented and/or arranged in combination in a single implementation and/or arrangement. Conversely, various features that are described in the context of a single implementation and/or arrangement can also be implemented and arranged in multiple implementations and/or arrangements separately or in any suitable sub combination. Moreover, although features can be described above as acting in certain combinations and even initially claimed as such, one or more features from a claimed combination can in some cases be excised from the combination, and the claimed combination can be directed to a sub combination or variation of a sub combination.

Additionally, features described with respect to particular headings can be utilized with respect to and/or in combination with illustrative arrangement described under other headings; headings, where provided, are included solely for the purpose of readability, and should not be construed as limiting any features provided with respect to such headings.

Similarly, while operations are depicted in the drawings in a particular order, this should not be understood as requiring that such operations be performed in the particular order shown or in sequential order, or that all illustrated operations be performed, to achieve desirable results. In some cases, the actions recited in the claims can be performed in a different order and still achieve desirable results. In addition, the processes depicted in the accompanying figures do not necessarily require the particular order shown, or sequential order, to achieve desirable results.

In certain circumstances, multitasking and parallel processing can be advantageous. Moreover, the separation of various system components in the implementations and/or arrangements described above should not be understood as requiring such separation in all implementations and/or arrangements, and it should be understood that the described program components and systems can generally be integrated together in a single software product or packaged into multiple software products.

Having now described some illustrative implementations, implementations, illustrative arrangements, and arrangements it is apparent that the foregoing is illustrative and not limiting, having been presented by way of example. In particular, although many of the examples presented herein involve specific combinations of method acts or system elements, those acts, and those elements can be combined in other ways to accomplish the same objectives. Acts, elements, and features discussed only in connection with one implementation and/or arrangement are not intended to be excluded from a similar role in other implementations or arrangements.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations and/or arrangements consisting of the items listed thereafter exclusively. In one arrangement, the systems and methods described herein consist of one, each combination of more than one, or all of the described elements, acts, or components.

Any references to implementations, arrangements, or elements or acts of the systems and methods herein referred to in the singular can also embrace implementations and/or arrangements including a plurality of these elements, and any references in plural to any implementation, arrangement, or element or act herein can also embrace implementations and/or arrangements including only a single element. References in the singular or plural form are not intended to limit the presently disclosed systems or methods, their components, acts, or elements to single or plural configurations. References to any act or element being based on any information, act or element can include implementations and/or arrangements where the act or element is based at least in part on any information, act, or element.

Any implementation disclosed herein can be combined with any other implementation, and references to “an implementation,” “some implementations,” “an alternate implementation,” “various implementation,” “one implementation” or the like are not necessarily mutually exclusive and are intended to indicate that a particular feature, structure, or characteristic described in connection with the implementation can be included in at least one implementation. Such terms as used herein are not necessarily all referring to the same implementation. Any implementation can be combined with any other implementation, inclusively or exclusively, in any manner consistent with the aspects and implementations disclosed herein.

Any arrangement disclosed herein can be combined with any other arrangement, and references to “an arrangement,” “some arrangements,” “an alternate arrangement,” “various arrangements,” “one arrangement” or the like are not necessarily mutually exclusive and are intended to indicate that a particular feature, structure, or characteristic described in connection with the arrangement can be included in at least one arrangement. Such terms as used herein are not necessarily all referring to the same arrangement. Any arrangement can be combined with any other arrangement, inclusively or exclusively, in any manner consistent with the aspects and arrangements disclosed herein.

References to “or” can be construed as inclusive so that any terms described using “or” can indicate any of a single, more than one, and all of the described terms.

Where technical features in the drawings, detailed description or any claim are followed by reference signs, the reference signs have been included for the sole purpose of increasing the intelligibility of the drawings, detailed description, and claims. Accordingly, neither the reference signs nor their absence have any limiting effect on the scope of any claim elements.

The systems and methods described herein can be embodied in other specific forms without departing from the characteristics thereof. The foregoing implementations and/or arrangements are illustrative rather than limiting of the described systems and methods. Scope of the systems and methods described herein is thus indicated by the appended claims, rather than the foregoing description, and changes that come within the meaning and range of equivalency of the claims are embraced therein.

It should be understood that no claim element herein is to be construed under the provisions of 35 U.S.C. § 112(f) unless the element is expressly recited using the phrase “means for.”

As used herein, the term “circuit” can include hardware structured to execute the functions described herein. In some embodiments, each respective “circuit” can include machine-readable media for configuring the hardware to execute the functions described herein. The circuit can be embodied as one or more circuitry components including, but not limited to, processing circuitry, network interfaces, peripheral devices, input devices, output devices, sensors. In some embodiments, a circuit can take the form of one or more analog circuits, electronic circuits (e.g., integrated circuits (IC), discrete circuits, system on a chip (SOC) circuits), telecommunication circuits, hybrid circuits, and any other type of “circuit.” In this regard, the “circuit” can include any type of component for accomplishing or facilitating achievement of the operations described herein. For example, a circuit as described herein can include one or more transistors, logic gates (e.g., NAND, AND, NOR, OR, XOR, NOT, XNOR), resistors, multiplexers, registers, capacitors, inductors, diodes, wiring.

The “circuit” can also include one or more processors communicatively coupled to one or more memory or memory devices. In this regard, the one or more processors can execute instructions stored in the memory or can execute instructions otherwise accessible to the one or more processors. In some embodiments, the one or more processors can be embodied in various ways. The one or more processors can be constructed in a manner sufficient to perform at least the operations described herein. In some embodiments, the one or more processors can be shared by multiple circuits (e.g., circuit A and circuit B can include or otherwise share the same processor which, in some example embodiments, can execute instructions stored, or otherwise accessed, via different areas of memory). Alternatively, or additionally, the one or more processors can be structured to perform or otherwise execute certain operations independent of one or more co-processors. In other example embodiments, two or more processors can be coupled via a bus to enable independent, parallel, pipelined, or multi-threaded instruction execution. Each processor can be implemented as one or more general-purpose processors, application specific integrated circuits (ASICs), field programmable gate arrays (FPGAs), digital signal processors (DSPs), or other suitable electronic data processing components structured to execute instructions provided by memory. The one or more processors can take the form of a single core processor, multi-core processor (e.g., a dual core processor, triple core processor, quad core processor), microprocessor. In some embodiments, the one or more processors can be external to the apparatus, for example the one or more processors can be a remote processor (e.g., a cloud based processor). Alternatively, or additionally, the one or more processors can be internal and/or local to the apparatus (e.g., internally located). In this regard, a given circuit or components thereof can be disposed locally (e.g., as part of a local server, a local computing system) or remotely (e.g., as part of a remote server such as a cloud based server). To that end, a “circuit” as described herein can include components that are distributed across one or more locations.

An exemplary system for implementing the overall system or portions of the embodiments might include a general purpose computing devices in the form of computers, including a processing unit, a system memory, and a system bus that couples various system components including the system memory to the processing unit. Each memory device can include non-transient volatile storage media, non-volatile storage media, non-transitory storage media (e.g., one or more volatile and/or non-volatile memories), etc. In some embodiments, the non-volatile media can take the form of ROM, flash memory (e.g., flash memory such as NAND, 3D NAND, NOR 3D NOR), EEPROM, MRAM, magnetic storage, hard discs, optical discs, etc. In other embodiments, the volatile storage media can take the form of RAM, TRAM, ZRAM, etc.

Combinations of the above are also included within the scope of machine-readable media. In this regard, machine-executable instructions include, for example, instructions and data which cause a general purpose computer, special purpose computer, or special purpose processing machines to perform a certain function or group of functions. Each respective memory device can be operable to maintain or otherwise store information relating to the operations performed by one or more associated circuits, including processor instructions and related data (e.g., database components, object code components, script components), in accordance with the example embodiments described herein.

It should also be noted that the term “input devices,” as described herein, can include any type of input device including, but not limited to, a keyboard, a keypad, a mouse, joystick, or other input devices performing a similar function. Comparatively, the term “output device,” as described herein, can include any type of output device including, but not limited to, a computer monitor, printer, facsimile machine, or other output devices performing a similar function.

It should be noted that although the diagrams herein can show a specific order and composition of method steps, it is understood that the order of these steps can differ from what is depicted. For example, two or more steps can be performed concurrently or with partial concurrence. Also, some method steps that are performed as discrete steps can be combined, steps being performed as a combined step can be separated into discrete steps, the sequence of certain processes can be reversed or otherwise varied, and the nature or number of discrete processes can be altered or varied. The order or sequence of any element or apparatus can be varied or substituted according to alternative embodiments. Accordingly, all such modifications are intended to be included within the scope of the present disclosure as defined in the appended claims. Such variations will depend on the machine-readable media and hardware systems chosen and on designer choice. It is understood that all such variations are within the scope of the disclosure. Likewise, software and web implementations of the present disclosure could be accomplished with standard programming techniques with rule-based logic and other logic to accomplish the various database searching steps, correlation steps, comparison steps and decision steps.

What is claimed is:

1. A method for verifying genetic samples using contextual metadata, comprising:
receiving at least one genetic sample;
identifying one or more contextual parameters associated with the at least one genetic sample;
obtaining one or more etches selected from a set of etches to encode the one or more contextual parameters; and
pooling the one or more etches with the at least one genetic sample in a sample environment, wherein pooling prevents binding of the one or more etches to the at least one genetic sample.

2. The method of claim 1, wherein the at least one genetic sample comprises a target genetic sample of an organism, and the method further comprising:
receiving, using the sample environment, the one or more etches pooled with the target genetic sample for sequencing;
decoding, based on a sequencing output, the one or more etches to identify metadata of the target genetic sample represented by the one or more contextual parameters;
executing an error-detection code on the sequencing output to detect at least one error associated with the metadata; and
executing an error-correction code on the sequencing output to correct the at least one error.

3. The method of claim 1, wherein the set of etches is stored in a library, and the method further comprising:
selecting, from the library, the one or more etches to encode the one or more contextual parameters based on an encoding scheme, wherein the encoding scheme assigns a combination of at least two etches to represent a corresponding contextual parameter; and
combining the at least two etches to encode the corresponding contextual parameter as a non-interfering genetic sequence.

4. The method of claim 1, further comprising:
detecting a contamination event based on identifying an etch pooled in the sample environment that encodes a contextual parameter associated with a genetic sample different from the at least one genetic sample.

5. The method of claim 1, further comprising:
storing the one or more contextual parameters encoded by the one or more etches in association with a physical or digital record corresponding to the at least one genetic sample.

6. The method of claim 1, wherein the one or more contextual parameters comprise at least one capture event parameter indicating information related to capture or collection of the at least one genetic sample and at least one processing event parameter indicating information related to processing, handling, or preparation of the at least one genetic sample.

7. The method of claim 1, wherein the set of etches is stored in a library, wherein the library maps a plurality of etches to corresponding metadata, and the method further comprising:
determining the one or more contextual parameters by comparing decoded values of the one or more etches to the corresponding metadata.

8. The method of claim 1, further comprising:
using the one or more contextual parameters encoded by the one or more etches to verify an identity of the at least one genetic sample based on comparing the one or more contextual parameters and expected metadata.

9. The method of claim 1, further comprising:
flagging, responsive to multiplexed sequencing of a plurality of genetic samples comprising the at least one genetic sample, an unexpected metadata combination associated with one or more of the plurality of genetic samples; and
resolving the unexpected metadata combination using one or more resolution techniques.

10. The method of claim 1, further comprising:
validating the one or more contextual parameters by applying a threshold to sequencing read counts associated with each of the one or more etches, wherein a contextual parameter is identified based on detecting a combination of sequences with read counts satisfying the threshold.

11. A system for verifying genetic samples using contextual metadata, comprising:
a set of etches; and
an encoding system configured to:
receive at least one genetic sample;
identify one or more contextual parameters associated with the at least one genetic sample;
obtain one or more etches selected from set of etches to encode the one or more contextual parameters; and
pool the one or more etches with the at least one genetic sample in a sample environment, wherein pooling prevents binding of the one or more etches to the at least one genetic sample.

12. The system of claim 11, wherein the at least one genetic sample comprises a target genetic sample of an organism, wherein the system further comprises:
a readout system configured to receive, using the sample environment, the one or more etches pooled with the target genetic sample for sequencing; and
a decoding system configured to:
decode, based on a sequencing output, the one or more etches to identify metadata of the target genetic sample represented by the one or more contextual parameters;
execute an error-detection code on the sequencing output to detect at least one error associated with the metadata; and
execute an error-correction code on the sequencing output to correct the at least one error.

13. The system of claim 11, wherein the set of etches is stored in a library, and wherein the encoding system is further configured to:
select, from the library, the one or more etches to encode the one or more contextual parameters based on an encoding scheme, wherein the encoding scheme assigns a combination of at least two etches to represent a corresponding contextual parameter; and
combine the at least two etches to encode the corresponding contextual parameter as a non-interfering genetic sequence.

14. The system of claim 11, further comprising:
a decoding system configured to detect a contamination event based on identifying an etch pooled in the sample environment that encodes a contextual parameter associated with a genetic sample different from the at least one genetic sample.

15. The system of claim 11, wherein the encoding system is further configured to:
store the one or more contextual parameters encoded by the one or more etches in association with a physical or digital record corresponding to the at least one genetic sample.

16. The system of claim 11, wherein the one or more contextual parameters comprise at least one capture event parameter indicating information related to capture or collection of the at least one genetic sample and at least one processing event parameter indicating information related to processing, handling, or preparation of the at least one genetic sample.

17. The system of claim 11, wherein the set of etches is stored in a library, wherein the library maps a plurality of etches to corresponding metadata, and the system further comprising:
a decoding system configured to determine the one or more contextual parameters by comparing decoded values of the one or more etches to the corresponding metadata.

18. The system of claim 11, further comprising:
a decoding system configured to use the one or more contextual parameters encoded by the one or more etches to verify an identity of the at least one genetic sample based on comparing the one or more contextual parameters and expected metadata.

19. A kit for verifying genetic samples using contextual metadata, the kit comprising:
a predefined set of etches configured to encode one or more contextual parameters associated with at least one genetic sample; and
a decoding reference database comprising mappings between the predefined set of etches and corresponding metadata values of the one or more contextual parameters.

20. A composition comprising:
at least one genetic sample; and
one or more etches selected to encode one or more contextual parameters associated with at least one genetic sample, wherein the one or more etches are non-covalently pooled with the at least one genetic sample in a sample environment.
